(19) United States
(12) Patent Application Publication
(10) Pub. No.: US 2020/0247761 A1
(43) Pub. Date: Aug. 6, 2020

(54) SULFONAMIDE OR AMIDE COMPOUNDS, COMPOSITIONS AND METHODS FOR THE PROPHYLAXIS AND/OR TREATMENT OF AUTOIMMUNE, INFLAMMATION OR INFECTION RELATED DISORDERS

(71) Applicant: TAIWANJ PHARMACEUTICALS CO., LTD, Zhubei City, Hsinchu County (TW)

(72) Inventors: Syaulan S. YANG, Chesterfield, MO (US); Kuang-Yuan LEE, Hsinchu City (TW); Meng-Hsien LIU, Toufen City (TW); Ming-Yu HSIAO, Shetou Township (TW); Huang-Kai PENG, Ershui Township (TW); Chiung-Wen WANG, Zhubei City (TW); Edwin SC WU, Cary, NC (US); Peter JS CHIU, Mill Creek, WA (US)

(73) Assignee: TAIWANJ PHARMACEUTICALS CO., LTD, Zhubei City, Hsinchu County (TW)

(21) Appl. No.: 16/488,517

(22) PCT Filed: Jan. 25, 2018
(86) PCT No.: PCT/US2018/015225

§ 371 Date: Aug. 23, 2019

Related U.S. Application Data
(60) Provisional application No. 62/462,927, filed on Feb. 24, 2017.

(51) Int. Cl. C07D 241/04; C07D 211/78; C07D 215/36; C07C 311/21; C07D 231/18; C07D 233/38; C07D 207/48; C07D 211/60; A61P 29/00

(52) U.S. Cl. CPC C07D 241/04; C07D 211/78; C07D 215/36; C07C 311/21; A61P 29/00; C07D 233/38; C07D 207/48; C07D 211/60; C07D 231/18

(57) ABSTRACT

The present invention related to novel sulfonamides or amides as TLR-4 antagonists, and pharmaceutical formulations containing the same and the methods of use thereof. Uses of the present novel sulfonamides or amides include, but are not limited to, the prophylaxis and/or treatment of autoimmune, inflammation, or infection related disorders.

### CROSS REFERENCE TO RELATED APPLICATION

This application claims the benefit of U.S. Provisional Application No. 62/462,927, filed on Feb. 24, 2017, the entirety of which is incorporated by reference herein.

### TECHNICAL FIELD

The present disclosure in general relates to novel compounds, which have been demonstrated to be toll-like receptor 4 (TLR-4) antagonists, are useful in the prophylaxis and/or treatment of diseases and/or disorders resulted from the activation of TLR-4.

### BACKGROUND

Toll-like receptors (TLRs) are transmembrane signaling receptors. There are at least 10 different TLRs in mammals, in which ligands and corresponding signaling cascades have been identified for some of them. For example, TLR2 is activated by the lipoprotein of bacteria (e.g., Escherichia coli), TLR3 is activated by double-stranded RNA, TLR4 is activated by lipopolysaccharide (i.e., LPS or endotoxin), TLR5 is activated by flagellin of motile bacteria (e.g., Listeria), TLR7 recognizes and responds to imiquimod, and TLR9 is activated by unmethylated CpG sequences of pathogen DNA. The stimulation of each of these TLRs leads to activation of the transcription factor NF-κB, and other signaling molecules that are involved in regulating the expression of cytokine genes, including those encoding mouse tumor necrosis factor-alpha (mTNF-α), interleukin-1 (IL-1), and certain chemokines. A number of diseases, disorders, and/or conditions have been demonstrated to be linked to TLRs, thus, TLR antagonists are regarded as potential drug candidates for treating diseases such as cancers, viral infections, inflammatory disease, and etc.

Unexpectedly, inventors of the present disclosure found that several novel compounds may antagonize the function of TLR-4. Accordingly, these novel compounds may be useful as lead compounds for the development of therapeutic agents for the prophylaxis and/or treatment of diseases and/or disorders mediated by the TLR-4.

### SUMMARY

The present disclosure is based on unexpected discovery that certain compounds are potent antagonists of TLR-4, thus are useful as lead compounds for the development of medicaments for the prophylaxis and/or treatment of diseases and/or disorders mediated by TLR-4, such as autoimmune diseases, inflammatory diseases and/or infectious diseases.

According to embodiments of the present invention, the present invention relates to a novel compound of Formula (I), or its enantiomer, diastereoisomer, solvate, hydrate, co-crystal, or pharmaceutically acceptable salt:

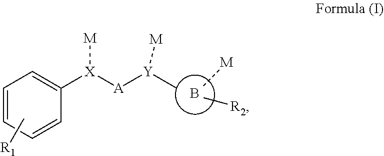

wherein:

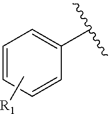

is a phenyl having at least one substituent of R₁ that is selected from the group consisting of H, alkyl, and halogen;
------ is nil or a single bond;
X and Y are independently nil, —NR′, or —CH₂—, in which R′ is H, alkyl, or a Michael acceptor;
A is —CO— or —SO₂—;
M is, H, alkyl, or a Michael acceptor;

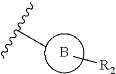

is a one or two fused ring system, a carbocyclyl or heterocyclyl, optionally having the Michael acceptor embedded therein or attached thereto and is optionally substituted with at least one substituent of R₂ that is selected from the group consisting of halogen, alkyl, haloalkoxy, —NO₂, —NRₐR_{b}, —NRₐCOR_{b}, —NRₐCOOR_{b}, —NRₐSO₂R_{b}, —NRₐSO₂NRₐR_{b}, —ORₐ, —CORₐ, —COORₐ, —SO₂Rₐ, and —SO₂NRₐR_{b};
Rₐ and R_{b} are independently H, C₁₋₂₀ alkyl, or aryl;
each alkyl and aryl is optionally substituted with at least one substituent selected from the group consisting of halogen, hydroxy, alkoxy, and phenyl; and
in cases where Y is nil, then B is not

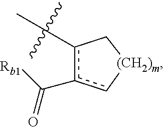

where [symbol] is a single or double bond, m is an integral between 1 to 4, and R_{b1} is H, alkyl, or alkoxy.

According to certain embodiments, in the Formula (I), the carbocyclyl is cycloalkyl, aryl, or phenyl. Preferably the carbocyclyl is cyclopropanyl or phenyl.

According to other embodiments, in the Formula (I), the heterocyclyl is heterocycloalkyl or heteroaryl optionally containing C, N, O, or S in the ring structure. Preferably, the heterocyclyl is a 5- or 6-membered monocyclic ring selected from the group consisting of furanyl, piperidinyl, piperazinyl, isoxazolyl, oxazolidine-2-one, and pyrrolidinyl. According to further embodiments, the heterocyclyl is a bi-cyclic ring of tetrahydroquinolinyl or quinoline.

In certain embodiments, the compound of Formula (I) can be the compound of Formula (II), or a pharmaceutically acceptable salt, solvate, hydrate, co-crystal, enantiomer, or diastereoisomer thereof:

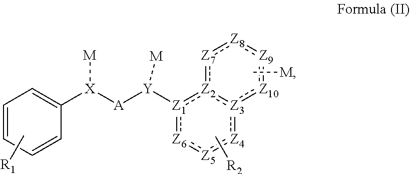

wherein:

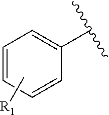

is a phenyl having at least one substituent of R₁ that is selected from the group consisting of H, alkyl, and halogen;
------ is nil or a single bond;
X and Y are independently nil, —NR′, or —CH₂—, in which R′ is H, alkyl, or M;
M is H, alkyl, or

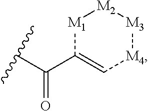

where ------ is as defined above, and M₁, M₂, M₃, and M₄ are independently nil, hydrogen, or methylene;
A is —CO— or —SO₂—;
Z₁ to Z₁₀ are independently nil, C, N, or O, and are taken together to form a carbocyclyl or heterocyclyl optionally having one substituent of M as defined above and at least one substituent of R₂ that is selected from the group consisting of halogen, alkyl, haloalkoxy, —NO₂, —NRₐR_{b}, —NRₐCOR_{b}, —NRₐCOOR_{b}, —NRₐSO₂R_{b}, —NRₐSO₂NRₐR_{b}, —ORₐ, —CORₐ, —COORₐ, —SO₂Rₐ, and —SO₂NRₐR_{b}, where [symbol] is a single or double bond, and Rₐ, and R_{b} are independently H, C₁₋₂₀ alkyl or aryl, in which each alkyl and aryl is optionally substituted with at least one substituent selected from the group consisting of halogen, hydroxy, alkoxy, and phenyl; and
in cases where Y is nil, then the carbocyclyl or heterocyclyl of Z₁ to Z₁₀ is not

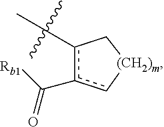

where [symbol] is as defined above, m is an integral between 1 to 4, and R_{b1} is H, alkyl, or alkoxy.

In certain embodiments, the compound of Formula (I) can be the compound of Formula (III), or a pharmaceutically acceptable salt, solvate, hydrate, co-crystal, enantiomer, or diastereoisomer thereof:

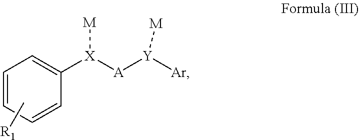

wherein:

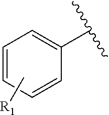

is a phenyl having at least one substituent of R₁ that is selected from the group consisting of H, alkyl, and halogen;
------ is nil or a single bond;
X and Y are independently nil, nitrogen, or carbon;
A is —CO or —SO₂—;
M is H, alkyl, or

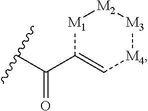

in which ------ is as defined above, and M₁, M₂, M₃, and M₄ are independently nil, hydrogen, or methylene;
Ar is phenyl or heteroaryl optionally substituted with at least one substituent selected from the group consisting of H, halogen, amine, nitro, alkyl, alkenyl, alkoxy, —COOR″, —SO₂R″, or —NHCOR″, where R″ is C₁-₂₀ alkyl or alkenyl; and
each alkyl and alkenyl is optionally substituted with at least one substituent selected from the group consisting of halogen, amine, nitro, or hydroxy.

A further aspect of the present disclosure is to provide a pharmaceutical composition for the prophylaxis and/or treatment of a subject having or suspected of having a disease and/or disorder mediated by TLR-4. The disease and/or disorder mediated by TLR-4 may be an autoimmune disease, an inflammatory disease or an infectious disease. The pharmacological composition comprises a therapeutically effective amount of the compound of Formula (I), (II) or (III); and a pharmaceutically acceptable carrier.

The compound of Formula (I), (II) or (III) is present at a level of about 0.1% to 99% by weight, based on the total weight of the pharmaceutical composition. In some embodiments, the compound of Formula (I), (II) or (III) is present at a level of at least 1% by weight, based on the total weight of the pharmaceutical composition. In certain embodiments, the compound of Formula (I), (II) or (III) is present at a level of at least 5% by weight, based on the total weight of the pharmaceutical composition. In still other embodiments, the compound of Formula (I), (II) or (III) is present at a level of at least 10% by weight, based on the total weight of the pharmaceutical composition. In still yet other embodiments, the compound of Formula (I), (II) or (III) is present at a level of at least 25% by weight, based on the total weight of the pharmaceutical composition.

The present disclosure also encompasses a method for the prophylaxis and/or treatment of a subject having a disease and/or disorder mediated by the activation of TLR-4. The method comprises the step of administering the present pharmaceutical composition to the subject, so as to ameliorate, mitigate and/or prevent the symptoms of the disease and/or disorder mediated by TLR-4. The disease and/or disorder mediated by TLR-4 may be an autoimmune disease, an inflammatory disease or an infectious disease.

Examples of the autoimmune disease treatable by the present method include, but are not limited to, multiple sclerosis, psoriasis, systemic lupus erythematosus (SLE), Type I diabetes mellitus, and Wegener's granulomatosis.

Examples of the inflammatory disease treatable by the present method include, but are not limited to, asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, systemic inflammatory response syndrome (SIRS), and ulcerative colitis.

Examples of the infectious disease preventable or treatable by the present method include, but are not limited to, bacterial, fungal and viral infections.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### DETAILED DESCRIPTION

In the following detailed description, for purposes of explanation, numerous specific details are set forth in order to provide a thorough understanding of the disclosed embodiments.

### 1. Definitions

When a range of values is listed, it is intended to encompass each value and sub-range within the range. For example, “C₁₋₄” is intended to encompass, C₁, C₂, C₃, C₄, C₁₋₄, C₁₋₃, C₁₋₂, C₂₋₄, C₂₋₃, and C₃₋₄. “C₁₋₂₀” is intended to encompass C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₁₋₂₀, C₁₋₁₉, C₁₋₁₈, C₁₋₁₇, C₁₋₁₆, C₁₋₁₅, C₁₋₁₄, C₁₋₁₃, C₁₋₁₂, C₁₋₁₁, C₁₋₁₀, C₁₋₉, C₁₋₈, C₁₋₇, C₁₋₆, C₁₋₅, C₁₋₄, C₁₋₃, C₁₋₂, C₂₋₂₀, C₂₋₁₉, C₂₋₁₈, C₂₋₁₇, C₂₋₁₆, C₂₋₁₅, C₂₋₁₄, C₂₋₁₃, C₂₋₁₂, C₂₋₁₁, C₂₋₁₀, C₂₋₉, C₂₋₈, C₂₋₇, C₂₋₆, C₂₋₅, C₂₋₄, C₂₋₃, C₃₋₂₀, C₃₋₁₉, C₃₋₁₈, C₃₋₁₇, C₃₋₁₆, C₃₋₁₅, C₃₋₁₄, C₃₋₁₃, C₃₋₁₂, C₃₋₁₁, C₃₋₁₀, C₃₋₉, C₃₋₈, C₃₋₇, C₃₋₆, C₃₋₅, C₃₋₄, C₄₋₂₀, C₄₋₁₉, C₄₋₁₈, C₄₋₁₇, C₄₋₁₆, C₄₋₁₅, C₄₋₁₄, C₄₋₁₃, C₄₋₁₂, C₄₋₁₁, C₄₋₁₀, C₄₋₉, C₄₋₈, C₄₋₂, C₄₋₆, C₄₋₅, C₅₋₂₀, l C₅₋₁₉, C₅₋₁₈, C₅₋₁₇, C₅₋₁₆, C₅₋₁₅, C₅₋₁₄, C₅₋₁₃, C₅₋₁₂, C₅₋₁₁, C₅₋₉, C₅₋₈, C₅₋₇, C₅₋₆, C₆₋₂₀, C₆₋₁₉, C₆₋₁₈, C₆₋₁₇, C₆₋₁₆, C₆₋₁₅, C₆₋₁₄, C₆₋₁₃, C₆₋₁₂, C₆₋₁₁, C₆₋₁₀, C₆₋₉, C₆₋₈, C₆₋₇, C₇₋₂₀, C₇₋₁₉, C₇₋₁₈, C₇₋₁₇, C₇₋₁₆, C₇₋₁₅, C₇₋₁₄, C₇₋₁₃, C₇₋₁₂, C₇₋₁₁, C₇₋₁₀, C₇₋₉, C₇₋₈, C₉₋₂₀, C₉₋₁₉, C₉₋₁₈, C₉₋₁₇, C₉₋₁₆, C₉₋₁₅, C₉₋₁₄, C₉₋₁₃, C₉₋₁₂, C₉₋₁₁, C₉₋₁₀, C₁₀₋₂₀, C₁₀₋₁₉, C₁₀₋₁₈, C₁₀₋₁₇, C₁₀₋₁₆, C₁₀₋₁₅, C₁₀₋₁₄, C₁₀₋₁₃, C₁₀₋₁₂, C₁₀₋₁₁, C₁₁₋₂₀, C₁₁₋₁₉, C₁₁₋₁₈, C₁₁₋₁₇, C₁₁₋₁₆, C₁₁₋₁₅, C₁₁₋₁₄, C₁₁₋₁₃, C₁₁₋₁₂, C₁₂₋₂₀, C₁₂₋₁₉, C₁₂₋₁₈, C₁₂₋₁₇, C₁₂₋₁₆, C₁₂₋₁₅, C₁₂₋₁₄, C₁₂₋₁₃, C₁₃₋₂₀, C₁₃₋₁₉, C₁₃₋₁₈, C₁₃₋₁₇, C₁₃₋₁₆, C₁₃₋₁₅, C₁₃₋₁₄, C₄₋₂₀, C₁₄₋₁₉, C₁₄₋₁₈, C₁₄₋₁₇, C₁₄₋₁₆, C₁₄₋₁₅, C₁₅₋₂₀, C₁₅₋₁₉, C₁₅₋₁₈, C₁₅₋₁₇, C₁₅₋₁₆, C₁₆₋₂₀, C₁₆₋₁₉, C₁₆₋₁₈, C₁₆₋₁₇, C₁₇₋₂₀, C₁₇₋₁₉, C₁₇₋₁₈, C₁₈₋₂₀, C₁₈₋₁₉, and C₁₉₋₂₀.

Unless otherwise indicated, the term “alkyl” means a straight chain, branched and/or cyclic (“cycloalkyl”) hydrocarbon having from 1 to 20 (e.g., 1 to 10, 1 to 9, 1 to 8, 1 to 7, 1 to 6, 1 to 5, 1 to 4, 1 to 3, 1 to 2, or 1) carbon atoms. Alkyl moieties having from 1 to 4 carbons (C₁₋₄ alkyl) are referred to as “lower alkyl.” Examples of alkyl groups include methyl, ethyl, propyl, isopropyl, n-butyl, t-butyl, isobutyl, 2-isopropyl-3-methyl butyl, pentyl, pentan-2-yl, hexyl, isohexyl, heptyl, heptan-2-yl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, and cyclohexyl. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is substituted C₂₋₁₀ alkyl. In some embodiments, cycloalkyl is a monocyclic, saturated carbocyclyl group having from 3 to 6 ring carbon atoms (“C₃₋₆ cycloalkyl”). In some embodiments, a cycloalkyl group has 5 to 6 ring carbon atoms (“C₅₋₆ cycloalkyl”). Examples of C₅₋₆ cycloalkyl groups include cyclopentyl (C₅) and cyclohexyl (C₆). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₆ cycloalkyl. In certain embodiments, the cycloalkyl group is substituted C₃₋₆ cycloalkyl.

Unless otherwise indicated, the term “alkenyl” means a straight chain, branched and/or cyclic (“cycloalkenyl”) hydrocarbon having from 2 to 20 (e.g., 2 to 10, 2 to 9, 2 to 8, 2 to 7, 2 to 6, 2 to 5, 2 to 4, 2 to 3, or 2) carbon atoms. The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted 2-propenyl.

“Carbocycle” or “carbocyclyl” as used herein refers to a saturated, partially unsaturated or aromatic ring having 3 to 7 carbon atoms as a monocycle, and 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo (4,5), (5,5), (5,6) or (6,6) system, or 9 or 10 ring atoms arranged as a bicyclo (5,6) or (6,6) system. Carbocycles includes aromatic and non-aromatic mono-, and bi-cyclic rings, whether fused, bridged, or spiro. Non-limiting examples of monocyclic carbocycles include the cycloalkyls group such as cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl or aryl groups such as phenyl, and the like. Thus, “carbocycle,” as used herein, encompasses but is not limited to “aryl”, “phenyl” and “biphenyl.”

The terms “heterocycle” or “heterocyclyl” as used herein refers to a 5- to 10-membered monocyclic or bicyclic ring, which may be saturated, partially unsaturated or aromatic containing 1, 2, or 3 heteroatoms selected from oxygen, nitrogen, and sulfur, which may be the same or different. Heterocycles includes aromatic and non-aromatic mono-, and bi-cyclic rings, whether fused, bridged, or spiro. In some embodiments of the invention “heterocycle” includes a “carbocycle” as defined herein, wherein one or more (e.g. 1, 2, or 3) carbon atoms are replaced with a heteroatom (e.g. O, N, or S). Unless otherwise specified, each instance of heterocyclyl is independently optionally substituted, i.e., unsubstituted (an “unsubstituted heterocyclyl”) or substituted (a “substituted heterocyclyl”) with one or more substituents. Examples of suitable substituents include halogen, alkyl, amino, nitro, hydroxyl, alkoxy, carbonyl, and carboxy. A non-limiting example of a carbonyl substituted heterocyclyl is

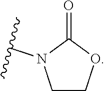 [SMILES: *N1CCOC1=O]

Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, thiadiazolinyl, and 1-azo-tetrahydrofuran-2-one. Exemplary 6-membered heterocyclyl groups containing one heteroatom include, without limitation, piperidinyl, tetrahydropyranyl, di hydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl.

Unless otherwise indicated, the term “aryl” means an aromatic ring or a partially aromatic ring system composed of carbon and hydrogen atoms. An aryl moiety may comprise multiple rings bound or fused together, such as a two fused ring system consisting of a phenyl ring fused with a 6-membered carbocyclyl or heterocyclyl groups, wherein the radical or point of attachment is on the aryl ring. Examples of aryl moieties include naphthyl, and phenyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is a substituted phenyl.

Unless otherwise indicated, the term “heteroaryl” means an aryl moiety wherein at least one of its carbon atoms has been replaced with a heteroatom (e.g., N, O or S). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃. The term “lower alkoxy” refers to —O-(lower alkyl), such as —OCH₃ and —OCH₂CH₃.

Unless otherwise indicated, the terms “halogen” encompass fluoro, chloro, bromo, and iodo.

The term “amino” refers to a moiety of the formula: —N(R)₂, wherein each instance of R is independently a substituent described herein, or two instances of R are connected to form substituted or unsubstituted heterocyclyl. In certain embodiments, the amino is unsubstituted amino (i.e., —NH₂). In certain embodiments, the amino is a substituted amino group, wherein at least one instance of R is not hydrogen.

An atom, moiety, or group described herein may be unsubstituted or substituted, as valency permits, unless otherwise provided expressly.

The term “optionally substituted” refers to substituted or unsubstituted. The term “substituted,” when used to describe a chemical structure or moiety, refers to a derivative of that structure or moiety wherein one or more of its hydrogen atoms is substituted with an atom, chemical moiety or functional group such as, but not limited to, —OH, —CHO, alkoxy, alkanoyloxy (e.g., —OAc), alkenyl, alkyl (e.g., methyl, ethyl, propyl, t-butyl), aryl, aryloxy, halo, or haloalkyl (e.g., —CCl₃, —CF₃, —C(CF₃)₃).

Unless otherwise indicated, one or more adjectives immediately preceding a series of nouns is to be construed as applying to each of the nouns. For example, the phrase “optionally substituted alky, cycloalkyl, heterocycloalkyl, aryl, or heteroaryl” has the same meaning as “optionally substituted alky, optionally substituted cycloalkyl, optionally substituted heterocycloalkyl, optionally substituted aryl, or optionally substituted heteroaryl.”

The term “Michael acceptor” refers to the substituent on an activated alkene in a Michael reaction, and is usually a ketone group making it an enone during the Michael reaction, which refers to nucleophilic addition of a carbanion to an α,β-unsaturated carbonyl compound. Michael acceptors are compounds containing unsaturated carbon-carbon bonds conjugated to electron withdrawing groups. Typically, they will contain α,β-ethylenic carbonyl group or its equivalent, such as 1,8-ethylenic aldehydes (e.g., acrolein. crotonaldehyde, or cinnamaldehyde), aliphatic α,β-ethylenic ketones, α,β-acetylenic ketones, aromatic α,β-ethylenic ketones, heterocyclic α,β-ethylenic ketones, cycloalkenones, acyl cycloalkenes, ρ-quinones, α,β-unsaturated nitriles, α,β-unsaturated amides, unsaturated imides (e.g., N-ethylmaleimide), α,β-ethylenic aliphatic esters, alicyclic (α,β-ethylenic esters, aromatic α,β-ethylenic esters, aromatic α,β-acetylenic esters, α,β-ethylenic nitro compounds, α,β-ethylenic sulfoxides and sulfones, α,β-ethylenic phosphonates, 2- and 4-vinylpyridines, fulvenes, and cyclopropane derivatives (e.g., ethyl 1-cyanocyclopropane-1-carboxylate).

According to certain embodiments of the present disclosure, the Michael acceptor has the structure of,

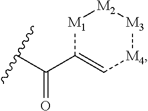

in which ------ is nil or a single bond, and M₁, M₂, M₃, and M₄ are independently nil, hydrogen, or methylene. In one preferred example, the Michael acceptor has the structure of

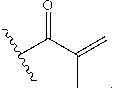 [SMILES: *C(=O)C(=C)C]

In another preferred example, the Michael acceptor has the structure of

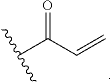 [SMILES: *C(=O)C=C]

The term “solvate” refers to forms of the compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences.

Unless otherwise indicated, “an effective amount” of a compound is an amount sufficient to provide a prophylactic or therapeutic benefit in the prevention, treatment, or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. A prophylactically effective amount of a compound is an amount of an agent that prevents or reduces the risk of occurrence of the disease or condition that is sought to be prevented. The term “effective amount” can encompass an amount that reduces the risk of occurrence of a disease or condition, improves overall therapy, reduces or avoids symptoms or causes of a disease or condition, or enhances the therapeutic efficacy of another therapeutic agent. Unless otherwise indicated, the terms “treat,” “treating”, and “treatment” contemplate an action that occurs while a patient is suffering from the specified disease or disorder, which reduces the severity of the disease or disorder, or one or more of its symptoms, or retards or slows the progression of the disease or disorder. The term “prevent,” “preventing,” “prevention,” and “prophylaxis” contemplate an action or any medical procedure with the purpose to prevent or suppress a disease and/or condition from occurring.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hem isulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable carrier” refers to a carrier, whether diluent or excipient, that is compatible with the other ingredients of a formulation and not deleterious to the recipient thereof.

The terms “administration of a composition” or “administering a composition” is defined to include an act of providing a compound or pharmaceutical composition of the present invention to the subject in need of treatment.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. Novel Compounds

The compounds as described herein can have the structure of Formula (I), or its pharmaceutically acceptable salt:

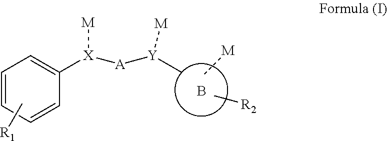

In some embodiments, they can be solvates, hydrates, polymorphs, co-crystals, stereoisomers, and prodrugs of Formula (I).

In Formula (I), ------ is nil or a single bond. M is H, alkyl, or a Michael acceptor. According to some preferred examples, the Michael acceptor has the structure of

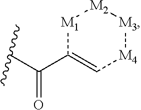

where ------ is as defined above, and M₁, M₂, M₃, and M₄ are independently nil, hydrogen, or methylene. In one example, the Michael acceptor has the structure of

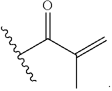 [SMILES: *C(=O)C(=C)C]

In another example, the Michael acceptor has the structure of

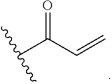

In Formula (I), X and Y may be nil, —NR′, or —CH₂—, in which R′ may be H, alkyl (e.g., C₁₋₄ alkyl), or the Michael acceptor as described above. Alternatively or in addition, A is —CO— or —SO₂—. In certain examples, X is —NR′—, where R′ is the Michael acceptor; Y is nil; and A is —SO₂—. In other examples, X is —NR′, where R′ is methyl; Y is carbon; and A is —SO₂—. In further example, X is —NR′, where R′ is H; Y is nil; and A is —CO—. In still further example, X is nil; Y is —NR′, where R′ is the Michael acceptor; and A is —SO₂—.

Alternatively or in addition, in Formula (I),

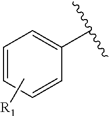

may be a phenyl having at least one substituent of R₁ that is selected from the group consisting of H, alkyl, and halogen. In certain examples,

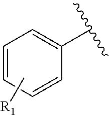

is a phenyl having one substituent, such as halogen. In other examples,

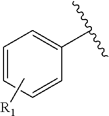

is a phenyl having two substituents, such as fluoro and chloro.

Alternatively or in addition, in Formula (I),

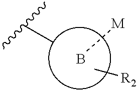

may be a one or two fused ring system, a carbocyclyl or heterocyclyl, optionally having the Michael acceptor (M) described above embedded therein or attached thereto. The carbocyclyl may be cycloalkyl, aryl, or phenyl. In some examples, the carbocylyl is cyclopropanyl or phenyl.

According to other embodiments, in the Formula (I), the heterocyclyl is heterocycloalkyl or heteroaryl optionally containing C, N, O, or S in the ring structure. Preferably, the heterocyclyl is a 5- or 6-membered monocyclic ring selected from the group consisting of furanyl, piperidinyl, piperazinyl, isoxazolyl, oxazolidine-2-one, and pyrrolidinyl. According to further embodiments, the heterocyclyl is a bi-cyclic ring of tetrahydroquinolinyl or quinoline.

Alternatively or in addition, in Formula (I), the carbocyclyl or heterocyclyl is optionally substituted with at least one substituent R₂ selected from the group consisting of halogen, alkyl, haloalkoxy, —NO₂, —NRₐR_{b}, —NRₐCOR_{b}, —NRₐCOOR_{b}, —NRₐSO₂R_{b}, —NRₐSO₂NRₐR_{b}, —ORₐ, —CORₐ, —COORₐ, —SO₂Rₐ, and —SO₂NRₐR_{b}. In Formula (I), Rₐ, and R_{b} are independently H, C₁₋₂₀ alkyl or aryl. Furthermore, each alkyl, Rₐ, and R_{b} are optionally substituted with at least one substituent selected from the group consisting of halogen, hydroxy, alkoxy, and phenyl.

Furthermore, in Formula (I), in cases where Y is nil, then B is not

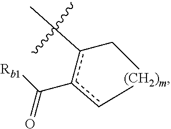

in which [symbol] is a single or double bond, m is an integral between 1 to 4, and R_{b1} is H, alkyl, or alkoxy.

In certain embodiments, the compound of Formula (I) can be of Formula (II), or a pharmaceutically acceptable salt, solvate, hydrate, co-crystal, enantiomer, or diastereoisomer thereof:

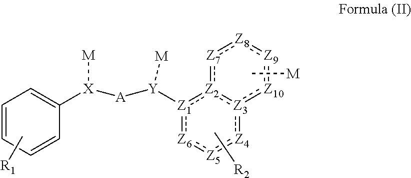

wherein:

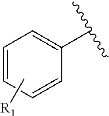

is a phenyl having at least one substituent of R₁ that is selected from the group consisting of H, alkyl, and halogen;
------ is nil or a single bond;
X and Y are independently nil, —NR′, or —CH₂—, in which R′ is H, alkyl, or M;
M is H, alkyl, or

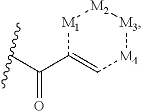

where ------ is as defined above, and M₁, M₂, M₃, and M₄ are independently nil, hydrogen, or methylene;
A is —CO— or —SO₂—;
Z₁ to Z₁₀ are independently nil, C, N, or O, and are taken together to form a carbocyclyl or heterocyclyl optionally having one substituent of M as defined above and at least one substituent of R₂ that is selected from the group consisting of halogen, alkyl, haloalkoxy, —NO₂, —NRₐR_{b}, —NRₐCOR_{b}, —NRₐCOOR_{b}, —NRₐSO₂R_{b}, —NRₐSO₂NRₐR_{b}, —ORₐ, —CORₐ, —COORₐ, —SO₂Rₐ, and —SO₂NRₐR_{b}, where [symbol] is a single or double bond, and Rₐ, and R_{b} are independently H, C₁₋₂₀ alkyl or aryl, in which each alkyl and aryl is optionally substituted with at least one substituent selected from the group consisting of halogen, hydroxy, alkoxy, and phenyl; and
in cases where Y is nil, then the carbocyclyl or heterocyclyl of Z₁ to Z₁₀ is not

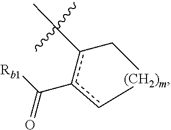

where [symbol] is as defined above, m is an integral between 1 to 4, and R_{b1} is H, alkyl, or alkoxy.

In certain embodiments, the compound of Formula (I) can be of Formula (III), or a pharmaceutically acceptable salt, solvate, hydrate, co-crystal, enantiomer, or diastereoisomer thereof:

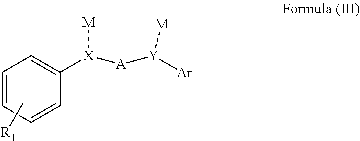

wherein:

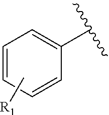

is a phenyl having at least one substituent of R₁ that is selected from the group consisting of H, alkyl, and halogen;
------ is nil or a single bond;
X and Y are independently nil, nitrogen, or carbon;
A is —CO or —SO₂—;
M is H, alkyl, or

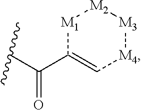

in which ------ is as defined above, and M₁, M₂, M₃, and M₄ are independently nil, hydrogen, or methylene;
Ar is phenyl or heteroaryl optionally substituted with at least one substituent selected from the group consisting of H, halogen, amine, nitro, alkyl, alkenyl, alkoxy, —COOR″, —SO₂R″, or —NHCOR″, where R″ is C₁₋₄ lower alkyl or alkenyl; and
each alkyl and alkenyl is optionally substituted with at least one substituent selected from the group consisting of halogen, amine, nitro, or hydroxy.

Exemplary preferred compounds of Formula (I), (II) and (III) include the compounds delineated in the following Table 1, but are not limited thereto.

TABLE 1

| Compound | |
|---|---|
| Number | Structure |
| 3 | 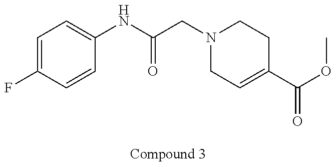 [SMILES: COC(=O)C1=CCN(CC(=O)Nc2ccc(F)cc2)CC1] |
| 6 | 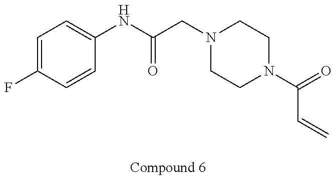 [SMILES: C=CC(=O)N1CCN(CC(=O)Nc2ccc(F)cc2)CC1] |
| 12 | 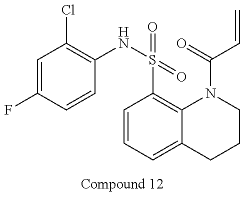 [SMILES: C=CC(=O)N1CCCc2cccc(S(=O)(=O)Nc3ccc(F)cc3Cl)c21] |
| 13 | 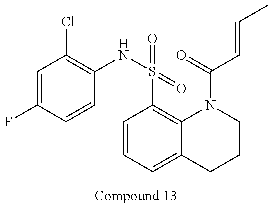 [SMILES: C/C=C/C(=O)N1CCCc2cccc(S(=O)(=O)Nc3ccc(F)cc3Cl)c21] |
| 15 | 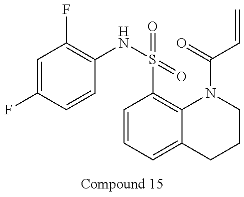 [SMILES: C=CC(=O)N1CCCc2cccc(S(=O)(=O)Nc3ccc(F)cc3F)c21] |
| 16 | 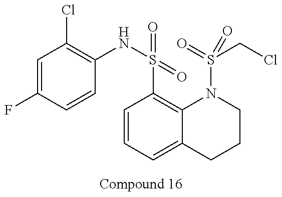 [SMILES: O=S(=O)(Nc1ccc(F)cc1Cl)c1cccc2c1N(S(=O)(=O)CCl)CCC2] |
| 19 | 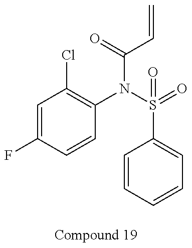 [SMILES: C=CC(=O)N(c1ccc(F)cc1Cl)S(=O)(=O)c1ccccc1] |
| 20 | 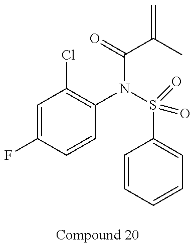 [SMILES: C=C(C)C(=O)N(c1ccc(F)cc1Cl)S(=O)(=O)c1ccccc1] |
| 22 | 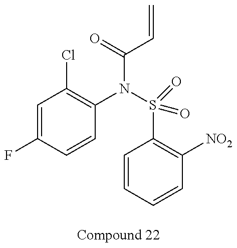 [SMILES: C=CC(=O)N(c1ccc(F)cc1Cl)S(=O)(=O)c1ccccc1[N+](=O)[O-]] |
| 24 | 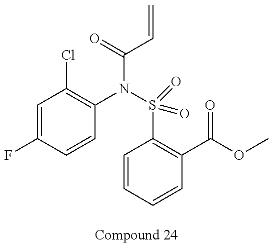 [SMILES: C=CC(=O)N(c1ccc(F)cc1Cl)S(=O)(=O)c1ccccc1C(=O)OC] |
| 26 | 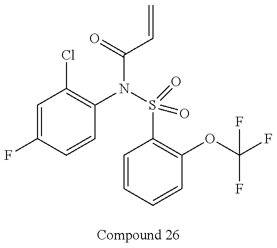 [SMILES: C=CC(=O)N(c1ccc(F)cc1Cl)S(=O)(=O)c1ccccc1OC(F)(F)F] |
| 28 | 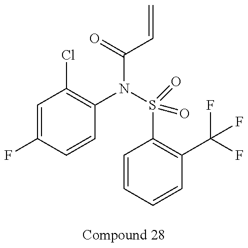 [SMILES: C=CC(=O)N(c1ccc(F)cc1Cl)S(=O)(=O)c1ccccc1C(F)(F)F] |
| 30 | 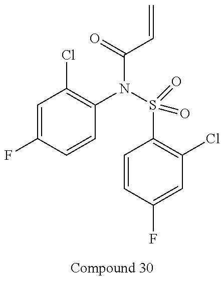 [SMILES: C=CC(=O)N(c1ccc(F)cc1Cl)S(=O)(=O)c1ccc(F)cc1Cl] |
| 32 | 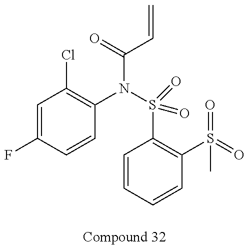 [SMILES: C=CC(=O)N(c1ccc(F)cc1Cl)S(=O)(=O)c1ccccc1S(C)(=O)=O] |
| 34 | 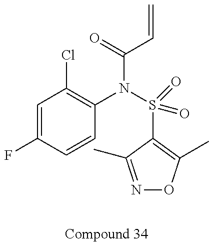 [SMILES: C=CC(=O)N(c1ccc(F)cc1Cl)S(=O)(=O)c1c(C)noc1C] |
| 36 | 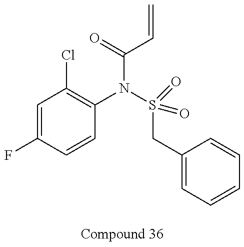 [SMILES: C=CC(=O)N(c1ccc(F)cc1Cl)S(=O)(=O)Cc1ccccc1] |
| 38 | 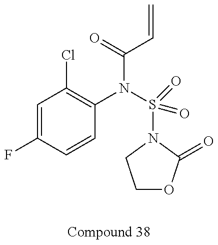 [SMILES: C=CC(=O)N(c1ccc(F)cc1Cl)S(=O)(=O)N1CCOC1=O] |
| 40 | 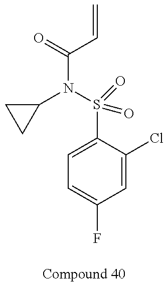 [SMILES: C=CC(=O)N(C1CC1)S(=O)(=O)c1ccc(F)cc1Cl] |
| 42 | 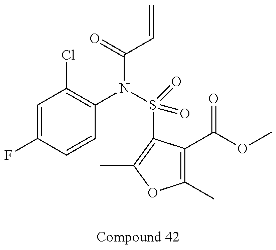 [SMILES: C=CC(=O)N(c1ccc(F)cc1Cl)S(=O)(=O)c1c(C)oc(C)c1C(=O)OC] |
| 44 | 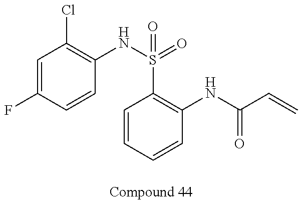 [SMILES: C=CC(=O)Nc1ccccc1S(=O)(=O)Nc1ccc(F)cc1Cl] |
| 45 | 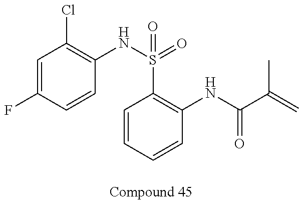 [SMILES: C=C(C)C(=O)Nc1ccccc1S(=O)(=O)Nc1ccc(F)cc1Cl] |
| 49 | 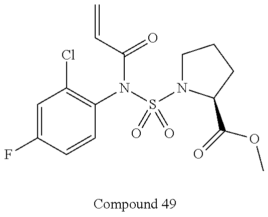 [SMILES: C=CC(=O)N(c1ccc(F)cc1Cl)S(=O)(=O)N1CCC[C@H]1C(=O)OC] |
| 50 | 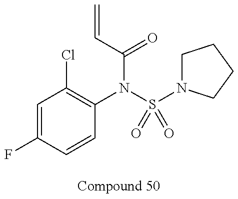 [SMILES: C=CC(=O)N(c1ccc(F)cc1Cl)S(=O)(=O)N1CCCC1] |
| 52 | 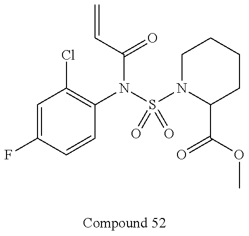 [SMILES: C=CC(=O)N(c1ccc(F)cc1Cl)S(=O)(=O)N1CCCCC1C(=O)OC] |
| 57 | 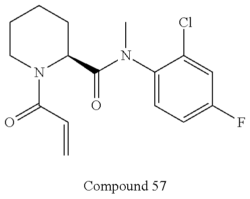 [SMILES: C=CC(=O)N1CCCC[C@H]1C(=O)N(C)c1ccc(F)cc1Cl] |
| 61 | 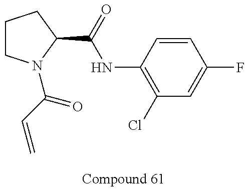 [SMILES: C=CC(=O)N1CCC[C@H]1C(=O)Nc1ccc(F)cc1Cl] |
| 65 | 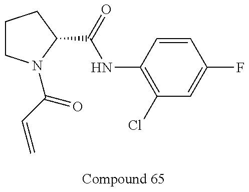 [SMILES: C=CC(=O)N1CCC[C@@H]1C(=O)Nc1ccc(F)cc1Cl] |
| 70 | 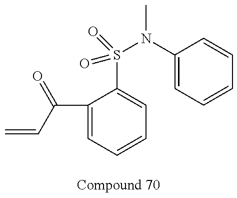 [SMILES: C=CC(=O)c1ccccc1S(=O)(=O)N(C)c1ccccc1] |

Each compound of the present invention contains one or more stereocenters, thus can exist as racemic mixtures of enantiomers or mixtures of diastereomers. This invention thus encompasses stereomerically pure forms of such compounds, as well as mixtures of those forms. Stereoisomers may be asymmetrically synthesized or resolved using standard techniques such as crystallization, chromatography, and the use of a resolving agent. One preferred way of separating enantiomers from a racemic mixture is by use of preparative high performance liquid chromatography (HPLC). Alternatively, the racemic may be separated into its enantiomers by reacting with an optically active form of a resolving agent in the presence of a solvent. Depending on the optical form of the resolving agent, one of the two enantiomers is separated out as an insoluble salt with high yield and high optical purity, while the opposite enantiomer remains in the solution. The present invention thus further encompasses stereoisomeric mixtures of compounds disclosed herein. It also encompasses configurational isomers of compounds disclosed herein (e.g., cis and trans isomers, whether or not involving double bonds), either in admixture or in pure or substantially pure form.

In certain embodiments, compounds of the present invention are the compounds described herein, and pharmaceutically acceptable salts, solvates, hydrates, co-crystals, and stereoisomers. In certain embodiments, compounds of the present invention are the compounds of any one of Formulae (I) to (III), and pharmaceutically acceptable salts, solvates, hydrates, co-crystals, and stereoisomers. In certain embodiments, compounds of the present invention are the compounds of any one of Formulae (I) to (III), and pharmaceutically acceptable salts thereof.

Each compound of the present invention described herein may be synthesized in accordance with methods set forth in the working examples of the present disclosure.

According to preferred embodiments of the present disclosure, the compound of Formulae (I) to (III) may suppress the NF-κB induced secretion of secreted alkaline phosphatase (SEAP), as well as lipopolysaccharide (LPS) induced mTNF-α release, both mechanisms are respectively linked to the activation of toll-like receptor 4 (TLR-4). Accordingly, the compound of Formulae (I) to (III) acts as an antagonist of TLR-4, thus may be useful as a lead compound for the development of a medicament suitable for the prophylaxis and/or treatment of diseases and/or disorders mediated by TLR-4.

### 3. Pharmaceutical Formulation

This invention encompasses pharmaceutical compositions for the prophylaxis and/or treatment of a disease and/or a disorder mediated by TLR-4. The pharmaceutical composition comprises a prophylactically or therapeutically effective amount of a compound of Formula (I), (H) or (III) of the present invention.

The compound of Formula (I), (II) or (III) is present at a level of about 0.1% to 99% by weight, based on the total weight of the pharmaceutical composition. In some embodiments, the compound of Formula (I), (II) or (III) is present at a level of at least 1% by weight, based on the total weight of the pharmaceutical composition. In certain embodiments, the compound of Formula (I), (II) or (III) s present at a level of at least 5% by weight, based on the total weight of the pharmaceutical composition. In still other embodiments, the compound of Formula (I), (II) or (III) is present at a level of at least 10% by weight, based on the total weight of the pharmaceutical composition. In still yet other embodiments, the compound of Formula (I), (II) or (III) is present at a level of at least 25% by weight, based on the total weight of the pharmaceutical composition.

According to embodiments of the present disclosure, the disease and/or disorder mediated by TLR-4 may be an autoimmune disease, an inflammatory disease or an infectious disease. Examples of the autoimmune disease treatable by the present pharmaceutical composition include, but are not limited to, multiple sclerosis, psoriasis, systemic lupus erythematosus (SLE), Type I diabetes mellitus, and Wegener's granulomatosis. Examples of the inflammatory disease treatable by the present pharmaceutical composition include, but are not limited to, asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, systemic inflammatory response syndrome (SIRS), and ulcerative colitis. Examples of the infectious disease treatable by the present pharmaceutical composition include, but are not limited to, bacterial, fungal and viral infections.

The formulation should suit the mode of administration. For example, oral administration requires enteric coatings to protect the compounds of this invention from degradation within the gastrointestinal tract. Similarly, a formulation may contain ingredients that facilitate delivery of the active ingredient(s) to the site of action. For example, compounds may be administered in liposomal formulations, in order to protect them from degradative enzymes, facilitate transport in circulatory system, and effect delivery across cell membranes to intracellular sites.

Similarly, poorly soluble compounds may be incorporated into liquid dosage forms (and dosage forms suitable for reconstitution) with the aid of solubilizing agents, emulsifiers and surfactants such as, but not limited to, cyclodextrins (e.g., α-cyclodextrin or β-cyclodextrin), and non-aqueous solvents, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, dimethyl sulfoxide (DMSO), biocompatible oils (e.g., cottonseed, groundnut, corn, germ, olive, castor, and sesame oils), glycerol, tetrahydrofurfuryl alcohol, polyethylene glycols, fatty acid esters of sorbitan, and mixtures thereof (e.g., DMSO:corn oil).

The composition, shape, and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art.

### 3.1 Oral Dosage Forms

Pharmaceutical compositions of the present invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or non-aqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Disintegrants may be incorporated in solid dosage forms to facility rapid dissolution. Lubricants may also be incorporated to facilitate the manufacture of dosage forms (e.g., tablets).

### 3.2 Parenteral Dosage Forms

Parenteral dosage forms can be administered to patients by various routes including, but not limited to, subcutaneous, intravenous (including bolus injection), intramuscular, and intra-arterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the present invention are well known to those skilled in the art. Examples include, but are not limited to: water; aqueous vehicles such as, but not limited to, sodium chloride solution, Ringer's solution, and Dextrose; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 3.3 Transdermal, Topical and Mucosal Dosage Forms

Transdermal, topical, and mucosal dosage forms include, but are not limited to, ophthalmic solutions, sprays, aerosols, creams, lotions, ointments, gels, solutions, emulsions, suspensions, or other forms known to one of skill in the art. Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the present invention. For example, penetration enhancers may be used to assist in delivering active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates may also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition

### 4. Kits

Also encompassed within the present disclosure is an article of manufacture or “kit,” containing materials useful for the treatment or prophylaxis of a disease and/or disorder mediated by TLR-4 in a subject.

In one embodiment, the kit comprises a container comprising the compound of the present disclosure. The kit is suitable for the treatment or prophylaxis of a disease and/or disorder mediated by TLR-4, such as autoimmune disease, inflammatory disease or infectious disease. Suitable containers include, for example, bottles, vials, syringes, blister pack, and etc. The container may be formed from a variety of materials such as glass, or plastic. The contain may hold a compound of the present disclosure or a pharmaceutical formulation thereof, in an amount effective for the treatment or prophylaxis of the disease and/or disorder mediated by TLR-4, and may have a sterile access port, for example, the container may be an intravenous solution bag or a vail having a stopper pierceable by a hypodermic injection needle). The kit may further comprise a label or package insert on or associated with the container. The label or package insert indicates that the composition is used for treating condition of choice. Alternatively or additionally, the kit may further comprise a second container comprising a pharmaceutically acceptable buffer, such as a phosphate-buffered saline, Ringer's solution or dextrose solution. It may further include other materials desirable from a commercial and user standpoint, including other buffers, diluents, filters, needles, and syringes.

The kit may further include directions for the administration of the compound of the present invention and, if present, the second formulation for treating or preventing the disease and/or disorder mediated by TLR-4. For example, if the kit comprises a first composition comprising the compound of the present disclosure, and a second pharmaceutical formulation, the kit may further include directions for the simultaneous, sequential, or separate administration of the first and second pharmaceutical compositions to a patient in need thereof.

In another embodiment, the kits are suitable for the delivery of solid oral forms of a compound of the present disclosure, such a kit includes, for example, a number of unit dosages. Such kits include card having the dosages oriented in the order of their intended use. An example of such kit is a “blister pack.” Blister packs are well known in the packaging industry and are widely used for packaging pharmaceutical unit dosage forms. If desired, an aid may be provided, for example, in the form of numbers, letters, or other markings or with a calendar insert, designating the days in the treatment schedule in which the dosage can be administered.

According to one embodiment, the Kit may include, at least, (a) a first container containing any of the present compound of Formula (I), (II) or (III); and optionally, (b) a second container containing a second therapeutic agent that is any of a known TLR-4 antagonist, an anti-inflammatory agent, an anti-biotic, or an immunosuppressant; and (c) a legend associated with the kit for instructing a user how to use the kit. The legend may be in a form of pamphlet, tape, CD, VCD or DVD.

### 5. Method of Use

The present invention encompasses a method for the prophylaxis and/or treatment of a subject having a disease and/or a disorder mediated by TLR-4. The method comprises the step of administering the present pharmaceutical composition, which comprises a prophylactically or therapeutically effective amount of any of the compound of Formula (I), (II) or (III) of the present disclosure, to the subject, so as to ameliorate, mitigate and/or prevent the symptoms of the disease and/or disorder mediated by TLR-4.

According to embodiments of the present disclosure, the disease and/or disorder mediated by TLR-4 may be an autoimmune disease, an inflammatory disease or an infectious disease. Examples of the autoimmune disease treatable by the present method include, but are not limited to, multiple sclerosis, psoriasis, systemic lupus erythematosus (SLE), Type I diabetes mellitus, and Wegener's granulomatosis. Examples of the inflammatory disease treatable by the present method include, but are not limited to, asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, systemic inflammatory response syndrome (SIRS), and ulcerative colitis. Examples of the infectious disease treatable by the present method include, but are not limited to, bacterial, fungal and viral infections.

The amount, route of administration and dosing schedule of the present pharmaceutical composition will depend upon factors such as the specific indication to be treated, prevented, or managed, and the age, sex and condition of the patient. The roles played by such factors are well known in the art, and may be accommodated by routine experimentation.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Materials and Methods

### Cell Culture

The HEK293 cells and RAW264.7 cells were respectively grown in the manufacturer's suggested medium supplemented with 10% heat-inactivated fetal bovine serum (FBS), 100 units/mL penicillin, and 100 μg/mL streptomycin, at 37° C. in a humidified 5% CO₂ incubator.

### Cell Viability Assay

RAW264.7 cells (1.6×10⁵ cells/well) or HEK 293 cells (2.5-5.0×10⁴ cells/well) were seeded in 96-well plate overnight and treated with different compounds for 16-24 hours. Cells' growth were determined by MTT-based colorimetric assays. Viability of the test cells treated with vehicle (0.5% DMSO) only was defined as 100% viable. Survival of cells after treatment with DETDs was calculated using the following formula: viable cell number (%)=OD₅₇₀ (treated cell culture)/OD₅₇₀ (vehicle control)×100.

### Example 1

### Preparation of the Compound of the Present Invention

### 1.1 Synthesis of Compound 3

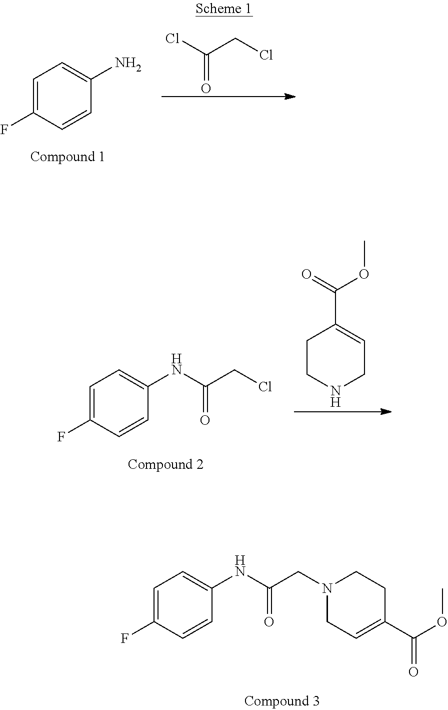

4-Fluoroaniline (Compound 1) (1.10 g, 10 mmole) and dichloromethane (35 ml) were charged into a reaction container equipped with stirred at room temperature. While stirring, chloroacetyl chloride (1.12 g, 10 mmole) was added slowly into the reaction container, and white precipitate appeared after 5 minutes, the mixture was then filtered and the organic layer was separated. The combined organic layer was sequentially extracted with dicholomethane (DCM, 35 ml×2), washed with saturated brine and dried over anhydrous Mg₂SO₄. Filtration and evaporation in vacuo gave the crude Compound 2 as white solid (1.14 g, the yield was 65%).

Compound 2 (188.6 mg, 1 mmole) was dissolved in acetonitrile (12 ml) with stirred at room temperature. Then, 4-pyridinecarboxylicacid (143.6 mg, 1 mmole) and triethylamine (330 mg, 3.3 mole) were sequentially added into the mixture. The reaction mixture was refluxed for 11 hours (monitor by TLC), then was neutralized with 5M NaOH (aq) (6 ml) to pH 8. The product was extracted with dichloromethane, and the organic layer was washed with brine, water and dried over MgSO₄. The solvent was evaporated and the crude residue was subjected to column chromatography (hexane/ethyl acetate=3:1) to give Compound 3 as a white solid (200 mg, 25% yield).

¹H-NMR (500 MHz, DMSO): δ 9.79 (s, 1H), 7.67-7.64 (m, 2H), 7.13 (t, 2H), 6.87 (s, 1H), 3.68 (s, 3H), 3.28-3.25 (m, 4H), 2.67 (t, 2H), 2.34 (s, 2H). ESI-MS m/z calcd for C₁₅H₁₇FN₂O₃ 292.12, found 293 [M+H]⁺.

### 1.2 Synthesis of Compound 6

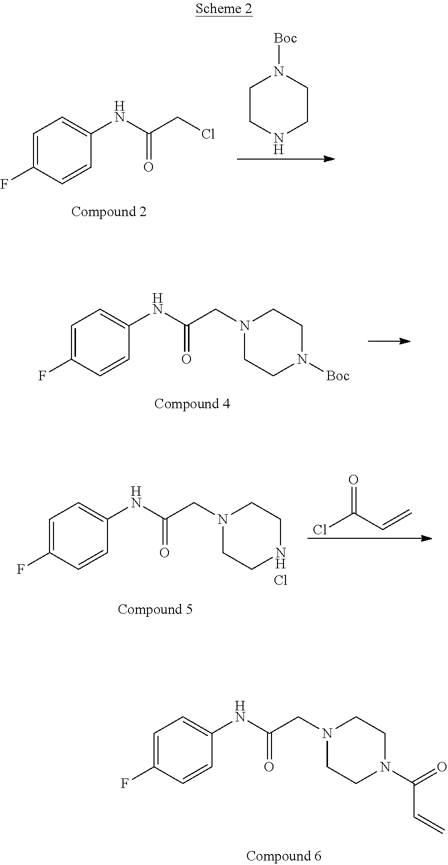

To a magnetically stirred solution of Compound 2 (173.6 mg, 1 mmole) in dichloromethane (15 mL) was added 1-Boc-piperazine (186.3 mg, 1 mmole), triethylamne (105.3 mg, 1 mmole) over water bath at 60° C. After the mixture was stirred and heated at 60° C. for 24 hours, the resulted mixture was neutralized to pH 8 and extracted with ethyl acetate (3×20 mL). The combined extracts were washed with brine, dried over anhydrous sodium sulfate, filtered, and concentrated. The residue thus obtained was purified by flash chromatography on silica gel with DCM/MeOH (10/1) to give Compound 4 (213 mg, 63% yield) as a light yellow liquid.

To a solution of mixture the crude Compound 4 and 2N HCl in ether at 25° C., stirred for 3 hours. The crude was concentrated to remove the HCl and thereby gave rise to the crude Compound 5. The crude compound 5 was used in the next step without further purification.

To a stirred solution of crude Compound 5 in CH₂Cl₂ (5 mL) under an atmosphere of nitrogen was added Et₃N (50.0 μl) and acyl chloride (35.0 μl) at 25° C., then stirred for 1 hour. The resulted mixture was extracted with EA and NaHCO₃ (aq). The extracts were combined and dried over anhydrous MgSO₄, filtered, and concentrated. The residue thus obtained was purified by flash chromatography on silica gel with EA to give Compound 6 (42.2 mg, 56% yield) as a white solid.

¹H-NMR (500 MHz, DMSO): δ 9.80 (s, 1H), 7.66-7.63 (m, 2H), 7.14 (t, 2H), 6.80 (dd, 1H), 6.11 (d, 1H), 5.66 (d, 1H), 3.61-3.58 (m, 4H), 3.31-3,29 (m, 4H), 3.16 (s, 2H). ESI-MS calcd for C₁₅H₁₈FN₃O₂ 291.14, found 292 [M+H]⁺.

### 1.3 Synthesis of Compounds 12, 13, 15, and 16

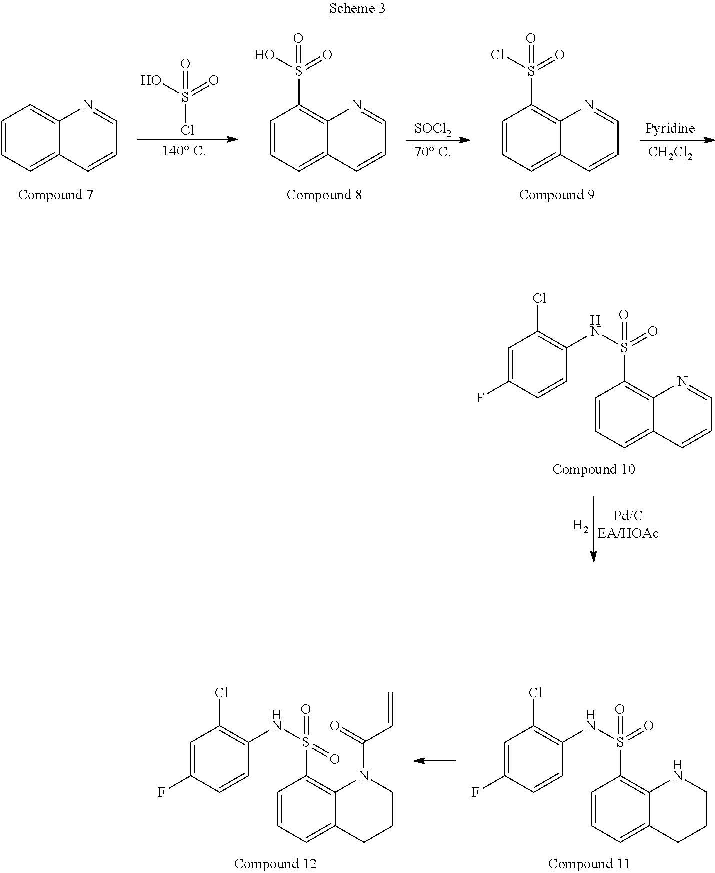

### 1.3.1 Synthesis of Compound 9

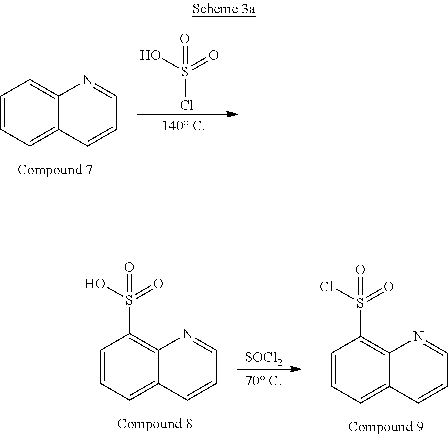

Chlorosulfonic acid (6.0 gram, 51.5 mmole) was charged into a container equipped with a magnetic stirrer. While stirring under nitrogen stream, quinoline (Compound 7) (1.0 g, 7.74 mmol) was added into the container under ice-cooling. While stirring, the temperature of the container was raised to 140° C. and reaction was allowed to proceed at the same temperature for 10 hours. Then, the temperature was reduced to 40° C., and thionyl chloride (2.0 g, 16.0 mmole) was added. While stirring, the temperature was raised to 70° C., allowed the reaction to proceed at the same temperature for 4 hours. After the reaction was completed, let the temperature returned to room temperature, then poured into ice-water, solid precipitate appeared immediately, stirred the mixture for 30 minutes, and then filtered, dried by vacuo, 1.7 g Compound 9 (yield: 99%) was obtained. m/z:228.0 (M+1).

### 1.3.2 Synthesis of Compound 10

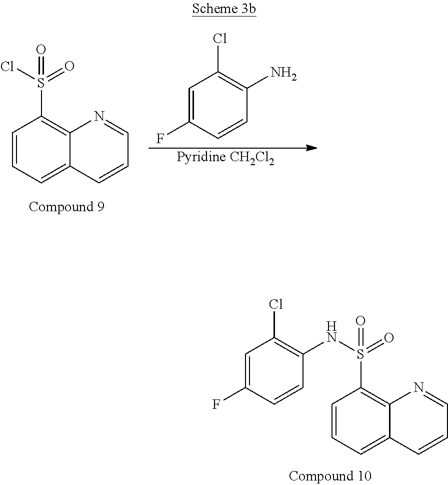

To a 150-mL round-bottomed flask containing Compound 9 (0.5 g, 2.2 mmol) in 15 mL of CH₂Cl₂, was added 2-chloro-4-fluoroaniline (0.33 g, 2.3 mmol) and pyridine (2 mL). The reaction mixture was stirred at room temperature for 2 hours. Evaporation of the solvents in vacuo gave an oily material, which was dissolved in EtOAc (15 mL), and the organic phase was separated and washed with 5% brine, dried over Na₂SO₄. Filtration and evaporation in vacuo gave title Compound 10 (0.7 g, 94%).

### 1.3.3 Synthesis of Compound 11

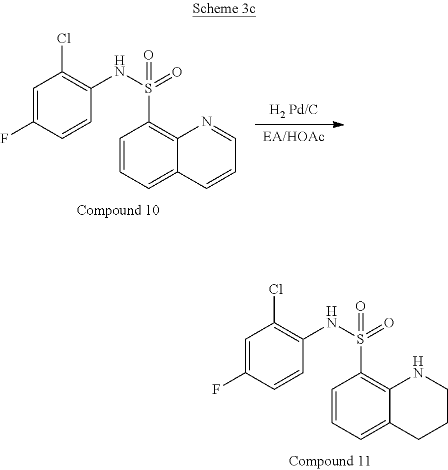 [SMILES: O=S(=O)(Nc1ccc(F)cc1Cl)c1cccc2cccnc12 ; O=S(=O)(Nc1ccc(F)cc1Cl)c1cccc2c1NCCC2]

To a 100-mL round-bottomed flask containing Compound 10 (0.7 g, 2.0 mmol) in EtOAc:EtOH (1:1, 10 mL), was added 10% Pd/C (0.1 g, 0.09 mmol, Aldrich) and AcOH (0.5 mL) under N₂. The reaction mixture was purged with H₂ and then stirred at room temperature for 18 hours under H₂ (H₂ balloon) atmosphere. After filtration through Celite, the filtrate was concentrated in vacuo to yield the title Compound 11, N-(2-chloro-4-fluorophenyl)-1,2,3,4-tetrahydro-8-quinolinesulfonamide (0.66 g, 93% yield).

### 1.3.4 Synthesis of Compound 12

Compound 12 was synthesized by following procedures set forth in either scheme 3d or 3e.

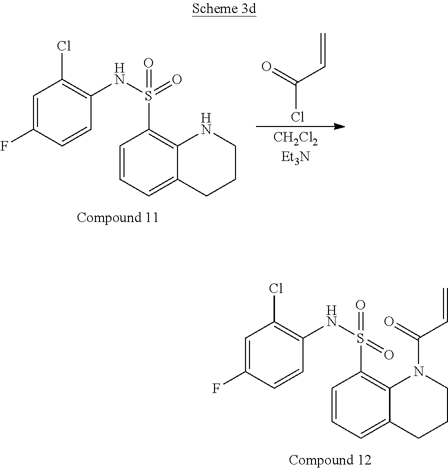

To a mixture of Compound 11 (0.1 g, 0.29 mmol), Et₃N (0.08 g, 0.75 mmol) and dichloromethane (10 mL) was added acryloyl chloride (0.03 g, 0.36 mmole) at 0° C. and the yellow solution was stirred at 25° C. for 12 hours. The solvent was removed and the residue was purified by column chromatography (Hexane/ethyl acetate=3:1). A white solid Compound 12 was obtained (0.052 g, 45% yield).

¹H-NMR (400 MHz, CDCl₃): δ 7.53 (d, 1H), 7.47-7.44 (m, 1H), 7.29-7.27 (m, 2H), 7.13-7.08 (m, 2H), 6.54-6.43 (m, 2H), 5.84 (dd, 1H), 5.66 (d, 1H), 3.41 (s, 2H), 2.79 (t, 2H), 1.95-1.89 (m, 2H). ESI-MS m/z calcd for C₁₈H₁₆CIFN₂O₃S 394.06, found 395.1 [M+H]⁺.

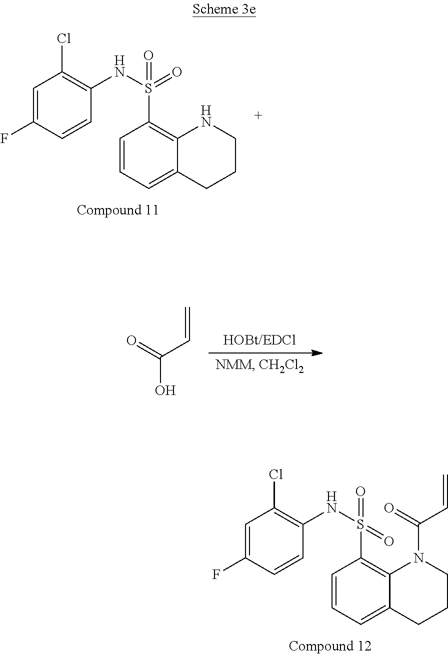

Equimolar amounts of acrylic acid and Compound 11 (0.1 g, 0.29 mmol) were dissolved in dichloromethane (10 mL). N-methylmorpholine (NMM) (1 eq) was added at 0° C. followed by EDC and HOBt after 15 minutes. The reaction mixture was stirred over night at room temperature, then washed with 1N HCl to pH 2. The product was extracted with dichloromethane, and the organic layer was washed with brine and water, then dried over MgSO₄. The solvent was evaporated and the crude residue was purified by column chromatography (hexane/ethyl acetate=3:1) to give a white powder Compound 12 (25% yield).

¹H-NMR (400 MHz, CDCl₃): δ 7.53 (d, 1H), 7.47-7.44 (m, 1H), 7.29-7.27 (m, 2H), 7.13-7.08 (m, 2H), 6.54-6.43 (m, 2H), 5.84 (dd, 1H), 5.66 (d, 1H), 3.41 (s, 2H), 2.79 (t, 2H), 1.95-1.89 (m, 2H). ESI-MS m/z calcd for C₁₈H₁₆CIFN₂O₃S 394.06, found 395.1 [M+H]⁺.

### 1.3.5 Synthesis of Compound 13

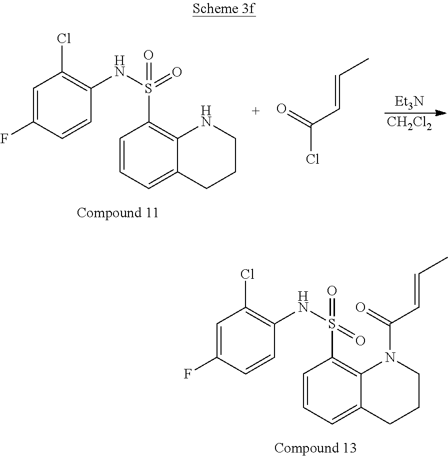

Compound 13 was produced by following Scheme 3f using compound 11 as the starting material. Yield of Compound 13 (light yellow solid) was 30%.

ESI-MS m/z calcd for C₁₉H₁₈CIFN₂O₃S 408.07, found 409.1 [M+H]⁺. ¹H-NMR (300 MHz, CDCl₃): δ 7.53 (d, 1H), 7.55 (dd, 1H), 7.29 (dd, 1H), 7.13-6.99 (m, 3H), 6.54-6.48 (m, 2H), 5.50 (d, 1H), 3.42-3.39 (m, 2H), 2.79 (t, 2H), 1.97-1.88 (m, 2H), 1.76 (s, 3H).

### 1.3.6 Synthesis of Compound 15

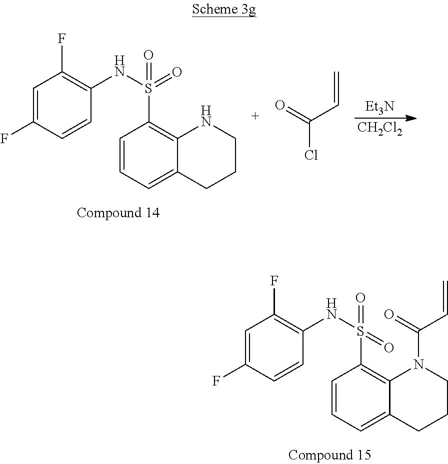

Compound 15 was produced by following Scheme 3g using Compound 14 as the starting material. Yield of Compound 15 (white solid) was 68%.

¹H-NMR (400 MHz, CDCl₃): δ 7.48 (d, 1H), 7.41-7.36 (m, 1H), 7.09 (d, 1H), 7.01-6.96 (m, 2H), 6.52 (t, 1H), 6.46-6.42 (m, 2H), 5.95 (dd, 1H), 5.67 (d, 1H), 3.41-3.38 (m, 2H), 2.79 (t, 2H), 1.89-1.81 (m, 2H). ESI-MS m/z calcd for C₁₈H₁₆F₂N₂O₃S 378.08, found 379.1 [M+H]⁺.

### 1.3.7 Synthesis of Compound 16

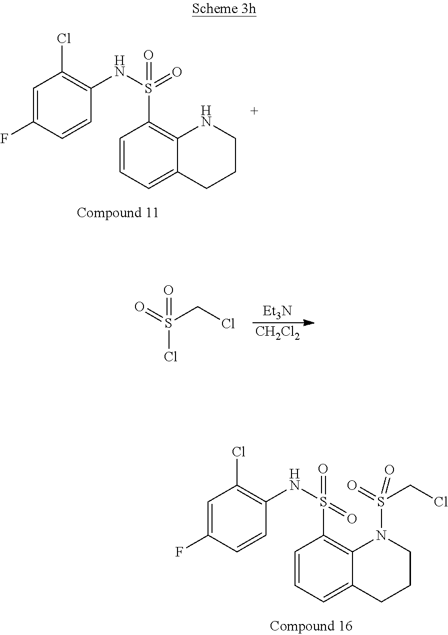

Compound 16 was produced by following Scheme 3f using Compound 11 as the starting material. Yield of Compound 16 (white solid) was 30% (mg).

¹H-NMR (400 MHz, CDCl₃): δ 7.64-7.60 (m, 1H), 7.19-7.17 (d, 1H), 7.12-7.08 (m, 2H), 7.05-7.00 (m, 1H), 6.42-6.38 (t, 1H), 6.26 (s, 1H), 5.45-5.42 (d, 1H), 4.97-4.93 (d, 1H), 3.35-3.26 (m, 2H), 2.76-2.73 (t, 2H), 1.89-1.81 (m, 2H). ESI-MS m/z calcd for C₁₆H₁₅Cl₂FN₂O₄S₂ 451.98, found 475.0 [M+Na]⁺.

### 1.4 Synthesis of Compounds 19, 20, 22, 24, 26, 28, 30, 32, 34, 36, 38, 40, and 42

### 1.4.1 Synthesis of Compound 19

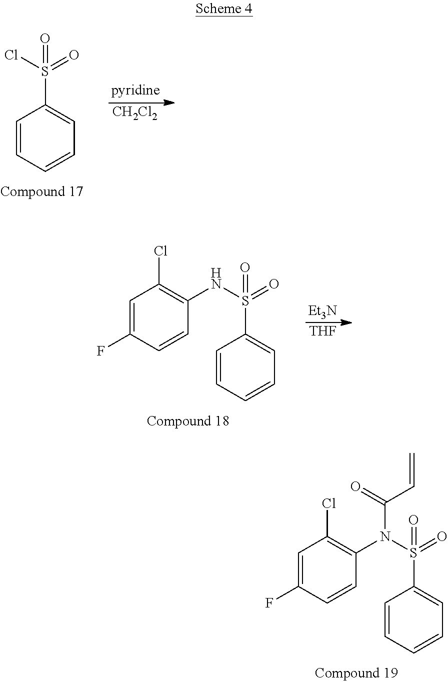

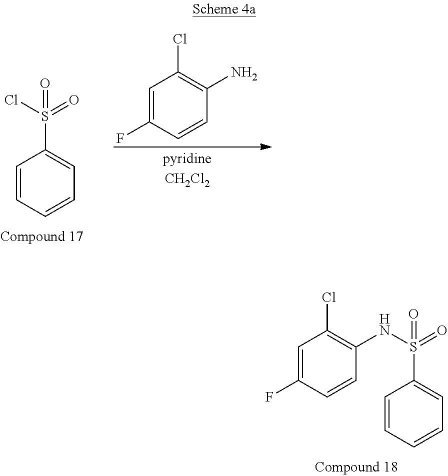 [SMILES: O=S(=O)(Cl)c1ccccc1 ; Nc1ccc(F)cc1Cl ; O=S(=O)(Nc1ccc(F)cc1Cl)c1ccccc1]

Compound 17 (1.76 g, 10 mmole) was added to a solution of the 2-chloro-4-fluoroaniline (1.45 g, 10 mmole) and pyridine (2 mL) in 15 mL of dichloromethane at 0° C.

The reaction mixture was allowed to warm to room temperature, and continued to stir at room temperature for 12 hours. The reaction mixture was quenched with water (20 mL), and extracted with dichloromethane (20 mL). The organic layer was separated and washed with HCl (0.4N, 10 mL) and brine, and concentrated to give the desired solid Compound 18 (2.8 g, 98%).

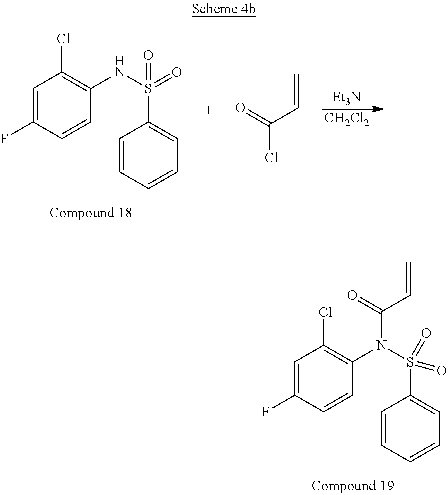

Acryloyl chloride (0.08 g, 0.84 mmol) was slowly added to a mixture of Et₃N (0.12 mL, 0.84 mmol) and Compound 18 (200 mg, 0.7 mmol) in CH₂Cl₂ (10 mL) at 0° C. The resulting solution was stirred at room temperature for 16 h. Then, the mixture was diluted with CH₂Cl₂, washed with aqueous NH₄Cl, and dried over anhydrous Na₂SO₄. The residue was submitted to flash chromatography (SiO₂, EtOAc/hexane, 20:80) to give the title Compound 19 (156 mg, 65%) as a white solid.

¹H-NMR (400 MHz, CDCl₃): δ 8.14 (d, 2H), 7.67 (t, 1H), 7.56 (t, 2H), 7.45 (dd, 1H), 7.30 (dd, 1H), 7.17-7.12 (m, 1H), 6.44 (dd, 1H), 5.80 (dd, 1H), 5.67 (dd, 1H). ESI-MS m/z calcd for C₁₅H₁₁CIFNO₃S 339.01, found 340.02 [M+H]⁺.

### 1.4.2 Synthesis of Compound 20

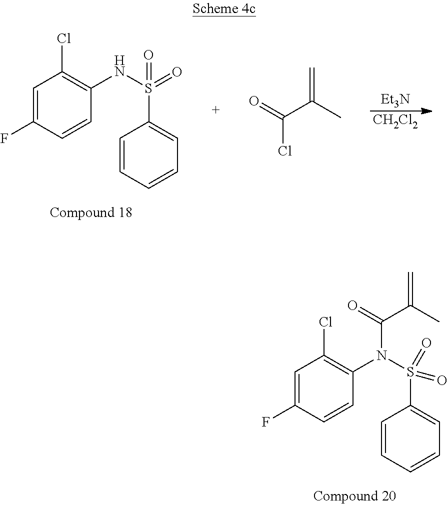

Compound 20 was produced by following Scheme 4c using Compound 18 as the starting material. Yield of Compound 20 was 20%. White solid

¹H-NMR (400 MHz, CDCl₃): δ 8.03 (d, 2H), 7.66 (t, 1H), 7.55-7.45 (m, 4H), 7.15 (d, 1H), 7.14-7.04 (m, 1H), 5.22 (d, 1H), 1.78 (s, 3H). ESI-MS m/z calcd for C₁₆H₁₃CIFNO₃S 353.03, found 354.04 [M+H]⁺.

### 1.4.3 Synthesis of Compound 22

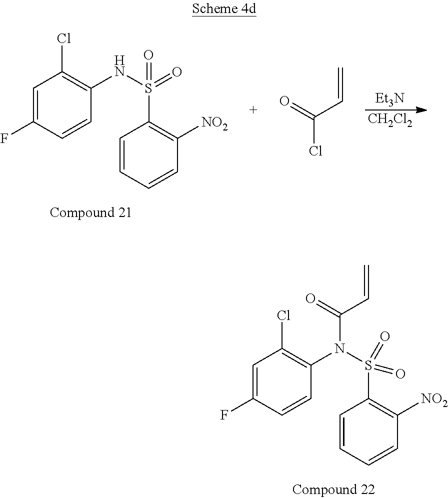

Compound 22 was produced by following Scheme 4d using Compound 21 as the starting material. Yield of Compound 22 was 35%. White solid.

¹H-NMR (400 MHz, CDCl₃): δ 8.64 (d, 1H), 7.80 (d, 2H), 7.71-7.64 (m, 2H), 7.29 (dd, 1H), 7.20-7.11 (m, 1H), 6.50 (d, 1H), 5.91 (dd, 1H), 5.75 (d, 1H). ESI-MS m/z calcd for C₁₅H₁₀CIFN₂O₅S 384.00, found 385.01 [M+H]⁺.

### 1.4.4 Synthesis of Compound 24

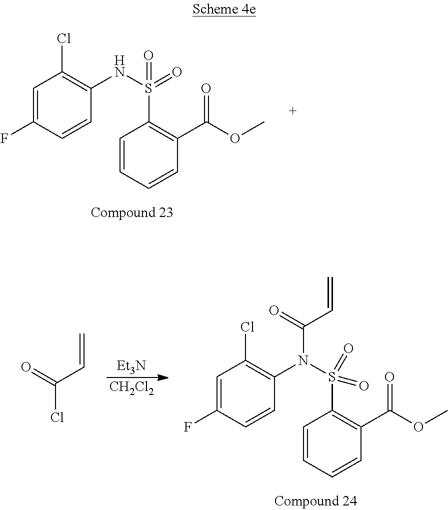

Compound 24 was produced by following Scheme 4e using Compound 23 as the starting material. Yield of Compound 24 was 17%. White solid.

¹H-NMR (300 MHz, CDCl₃): δ 8.50 (d, 1H), 7.70-7.62 (m, 3H), 7.54-7.51 (m, 1H), 7.26 (t, 1H), 7.13 (td, 1H), 6.48 (d, 1H), 5.90 (dd, 1H), 5.69 (d, 1H), 3.70 (s, 3H). ESI-MS m/z calcd for C₁₇H₁₃CIFNO₅S 397.02, found 420.0 [M+Na]⁺.

### 1.4.5 Synthesis of Compound 26

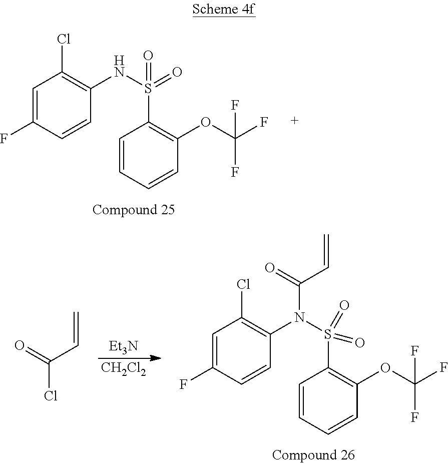

Compound 26 was produced by following Scheme 4f using Compound 25 as the starting material. Yield of Compound 26 was 16%. White solid.

¹H-NMR (300 MHz, CDCl₃): δ 8.78-8.74 (m, 1H), 7.57 (dt, 2H), 7.47 (td, 1H), 7.26-7.22 (m, 1H), 7.15 (td, 1H), 6.46 (d, 1H), 5.90 (dd, 1H), 5.72 (d, 1H). ESI-MS m/z calcd for C₁₆H₉CIF₅NO₃S 424.99, found 426.1 [M+H]⁺.

### 1.4.6 Synthesis of Compound 28

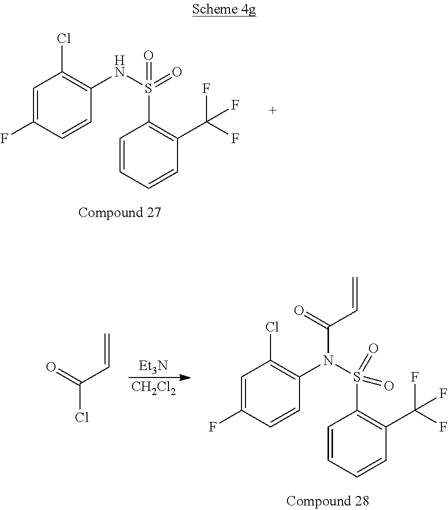

Compound 28 was produced by following Scheme 4g using Compound 27 as the starting material. Yield of Compound 28 was 81%. White solid.

¹H-NMR (300 MHz, CDCl₃): δ 8.40 (dd, 1H), 7.72 (td, 1H), 7.57 (q, 1H), 7.52 (t, 1H), 7.41 (d, 1H), 7.30 (dd, 1H), 7.17-7.10 (m, 1H), 6.44 (dd, 1H), 5.90 (dd, 1H), 5.70 (dd, 1H). ESI-MS m/z calcd for C₁₆H₁₀CIF₄NO₄S 423.00, found 424.00 [M+H]⁺.

### 1.4.7 Synthesis of Compound 30

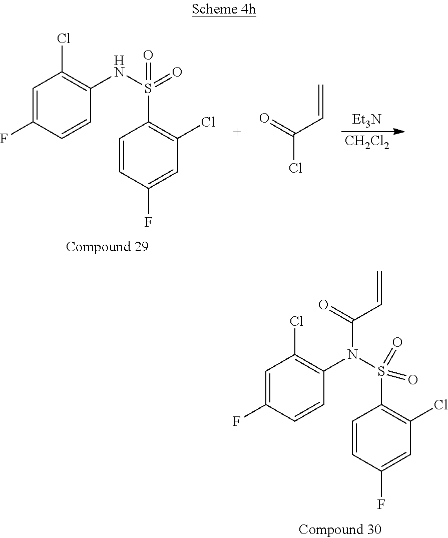

Compound 30 was produced by following Scheme 4h using compound 29 as the starting material. Yield of Compound 30 was 32%. White solid.

¹1-1-NMR (400 MHz, CDCl₃): δ 8.51-8.47 (m, 1H), 7.66-7.63 (m, 1H), 7.31-7.12 (m, 4H), 6.46 (d, 1H), 5.90 (dd, 1H), 5.72 (d, 1H). ESI-MS m/z calcd for C₁₅H₉Cl₂F₂NO₃S 390.96, found 392.00 [M+H]⁺.

### 1.4.8 Synthesis of Compound 32

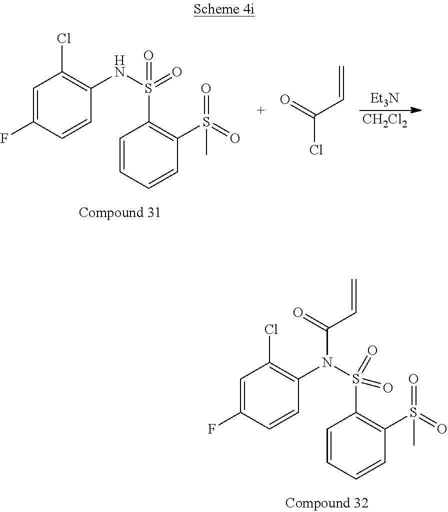

Compound 32 was produced by following Scheme 4i using compound 31 as the starting material. Yield of Compound 32 was 44%. White solid.

¹H-NMR (400 MHz, CDCl₃): δ 8.64 (d, 1H), 8.40 (d, 1H), 7.91-7.81 (m, 3H), 7.30 (d, 1H), 7.19-7.10 (m, 1H), 6.42 (d, 1H), 5.95 (dd, 1H), 5.70 (d, 1H), 3.40 (s, 3H). ESI-MS m/z calcd for C₁₆H₁₃CIFNO₅S₂ 416.99, found 418.00 [M+H]⁺.

### 1.4.9 Synthesis of Compound 34

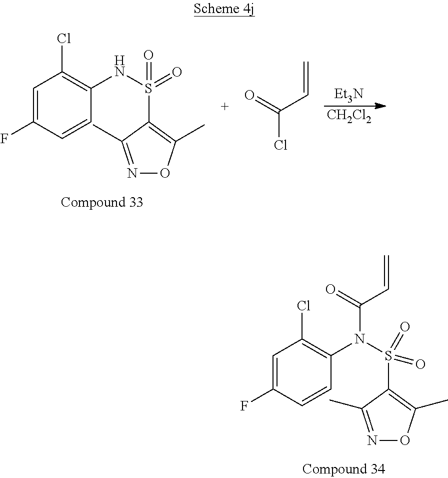

Compound 34 was produced by following Scheme 4j using compound 33 as the starting material. Yield of Compound 34 was 88%. White solid.

¹H-NMR (400 MHz, CDCl₃): δ 7.50 (dd, 1H), 7.31 (dd, 1H), 7.20-7.15 (m, 1H), 6.50 (d, 1H), 5.84-5.70 (m, 2H), 2.56 (s, 3H), 2.38 (s, 3H). ESI-MS m/z calcd for C₁₄H₁₂CIFN₂O₄S 358.02, found 359.78 [M+H]⁺.

### 1.4.10 Synthesis of Compound 36

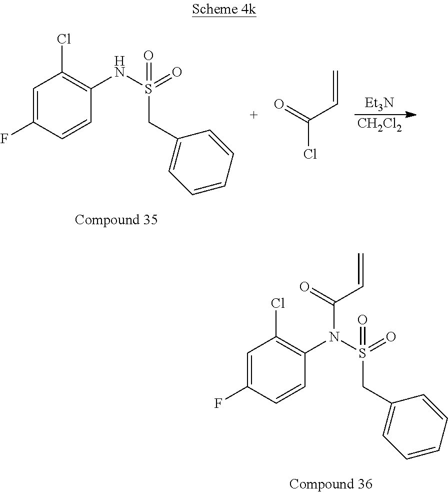

Compound 36 was produced by following Scheme 4k using compound 35 as the starting material. Yield of Compound 36 was 49%. White solid.

¹H-NMR (400 MHz, CDCl₃): δ 7.50-7.40 (m, 5H), 7.21 (dd, 1H), 6.86-6.78 (m, 1H), 6.62 (d, 1H), 6.00 (dd, 1H), 5.80-5.64 (m, 2H), 5.30 (d, 1H), 4.71 (d, 1H). ESI-MS m/z calcd for C₁₆H₁₃ClFNO₃S 353.03, found 354.04 [M+14]⁺.

### 1.4.11 Synthesis of Compound 38

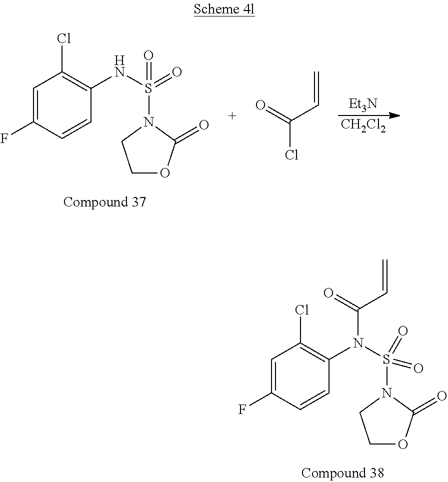

Compound 38 was produced by following Scheme 41 using compound 37 as the starting material. Yield of Compound 38 was 30%. White solid.

¹H-NMR (400 MHz, CDCl₃): δ 7.91 (dd, 1H), 7.29 (dd, 1H), 7.18 (dd, 1H), 6.56 (dd, 1H), 5.80-5.77 (m, 2H), 4.73 (q, 1H), 4.57-4.51 (m, 2H), 4.28 (q, 1H). ESI-MS m/z calcd for C₁₂H₁₀ClFN₂O₅S 348.00, found 371.00 [M+Na]⁺.

### 1.4.12 Synthesis of Compound 40

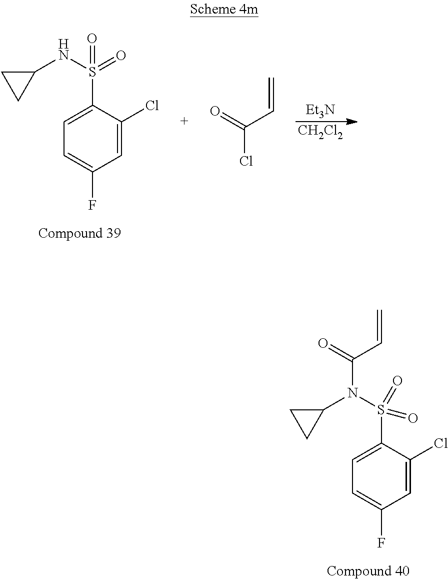

Compound 40 was produced by following Scheme 4m using compound 39 as the starting material. Yield of Compound 40 was 7%. White solid.

¹H-NMR (400 MHz, CDCl₃): δ 8.30-8.26 (m, 1H), 7.29-7.16 (m, 2H), 6.86-6.78 (m, 1H), 6.35 (d, 1H), 5.82 (d, 1H), 2.95-2.91 (m, 1H), 1.26-1.20 (m, 2H), 1.18-1.11 (m, 2H). ESI-MS m/z calcd for C₁₂H₁₁ClFNO₃S 303.01, found 326.0 [M+Na]⁺.

### 1.4.13 Synthesis of Compound 42

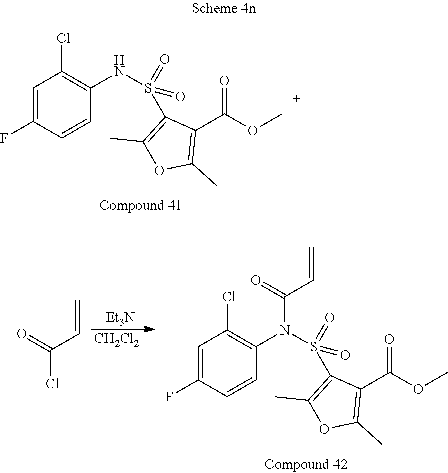

Compound 42 was produced by following Scheme 4n using compound 41 as the starting material. Yield of Compound 42 was 23%. White solid.

¹H-NMR (400 MHz, CDCl₃): δ 7.93-7.90 (m, 1H), 7.26-7.24 (m, 1H), 7.17-7.12 (m, 1H), 6.45 (d, 1H), 5.99 (t, 1H), 5.68 (d, 1H), 3.69 (s, 3H), 2.69 (s, 3H), 2.50 (s, 3H). ESI-MS m/z calcd for C₁₇H₁₅ClFNO₆S 415.03, found 438.1 [M+Na]⁺.

### 1.5 Synthesis of Compounds 44 and 45

### 1.5.1 Synthesis of Compound 44

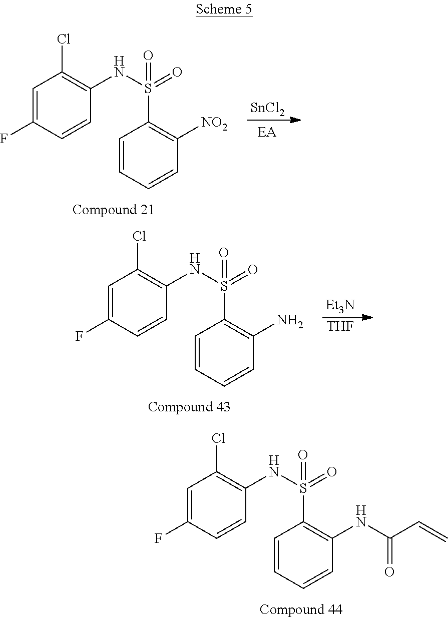

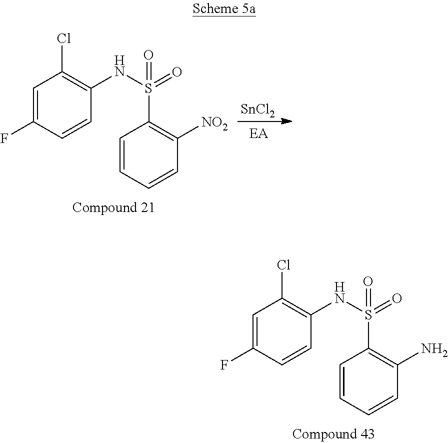

A solution of the Compound 21 (3.0 g, 9.1 mmol) and SnCl₂ (8.63 g, 45.5 mmol) in EtOAc (120 mL) was refluxed for 15 hours until TLC (EtOAc/hexane, 1:3) indicated that the reaction was completed. The reaction mixture was poured into a 100 mL beaker and diluted with 20 mL of EtOAc, followed by the addition of potassium carbonate and deionized water (20 mL), and the two layers were stirred for 30 minutes. The milky suspension was filtered through a short bed of celite and the organic layer was separated. The combined organic layer was sequentially treated with saturated brine and anhydrous Na₂SO₄. Purification by flash column chromatography using EtOAc: hexane (1:3) afforded desired Compound 43, 1.88 g, 69% yield.

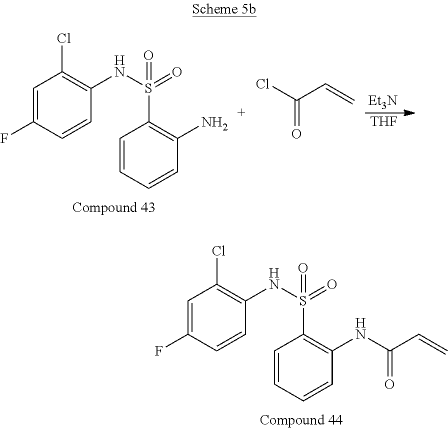

Compound 44 was produced by following Scheme 5b using Compound 43 as the starting material. Yield of Compound 44 was 60%. White solid.

¹H-NMR (400 MHz, CDCl₃): δ 9.77 (s, 1H), 8.56 (d, 1H), 7.80 (d, 1H), 7.71 (t, 1H), 7.43 (dd, 1H), 7.31 (dd, 1H), 7.20-7.11 (m, 2H), 6.50 (dd, 1H), 5.80-5.70 (m, 2H).

### 1.5.2 Synthesis of Compound 45

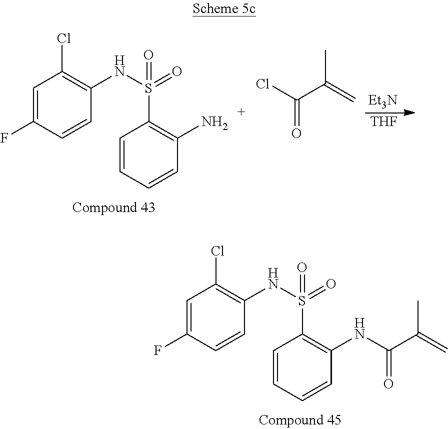

Compound 45 was produced by following Scheme 5c using compound 43 as the starting material. Yield of Compound 44 was 60%. White solid.

¹H-NMR (400 MHz, CDCl₃): δ 9.66 (s, 1FI), 8.55 (d, 1H), 7.67 (dd, 1H), 7.62 (t, 1H), 7.50 (dd, 1H), 7.19 (dd, 1H), 7.16-7.08 (in, 2H), 5.30 (s, 114), 5.28 (s, 1H), 2.18 (s, 3H).

### 1.6 Synthesis of Compounds 49, 50, and 52

### 1.6.1 Synthesis of Compound 49

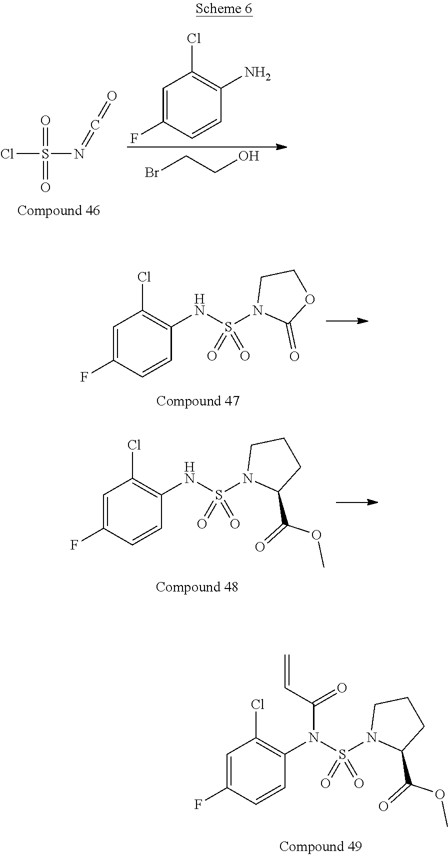

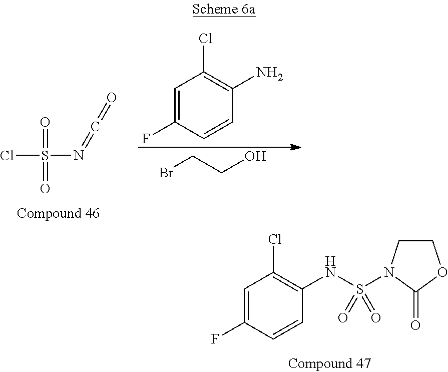

To a 250-mL glass-lined reactor initially charged with dichloromethane (10 mL) was added chlorosulfonyl isocyanate (2.25 g, 15.9 mmol) at room temperature and under a nitrogen atmosphere. The reaction mixture was cooled to about 1° C., and a solution of 2-bromoethanol (2.0 g, 16 mmol) in dichloromethane (10 mL) was slowly added over 10 minutes to keep the reaction temperature between 0 and 10° C. Continued to stir the reaction mixture at the same temperature for at least 30 minutes. A mixture of 2-chloro-4-fluoroaniline (2.57 g, 17.7 mmol) and triethylamine (3.55 g, 35 mmol) in dichloromethane (10 mL) was then added at the addition rate that the reaction temperature was maintained between 0-10° C. The solution was warmed to room temperature. Aqueous hydrochloric acid (0.2 N, 10mL) was then added, and the pH of the reaction mixture was adjusted to about 2 by the addition of HCl. The reaction mixture was extracted with dichloromethane and dried over MgSO₄, filtered, concentrated. Purification by flash column chromatography using EtOAc: hexane (1:2) afforded desired compound 3.74 g, 80% yield.

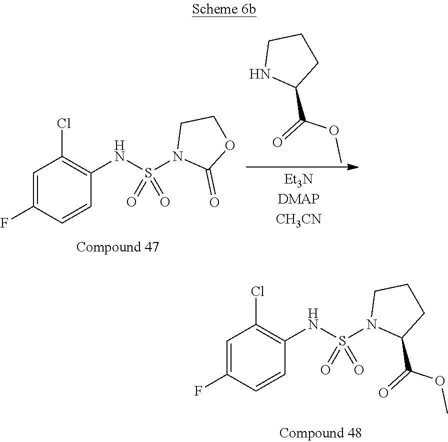

A separate flame dried flask was charged with the appropriate oxazolidinone substrate (0.5 g, 1.7 mmole), 4-dimethylaminopyridine (0.05 g, 0.4 mmole), and a stirbar, then was evacuated and backfed with N₂. Acetonitrile was added, followed by Et₃N (0.52 g, 5.1 mmole), and then the reaction vessel was placed in an oil bath at 75° C. The H-PRO-OMe HCl (0.34 g, 2.0 mmole) was added and the resulting mixture was stirred at 75° C. overnight.

The mixture was cooled to room temperature, solvent was removed via rotary evaporation, and the residue was partitioned between CH₂Cl₂ and 2N HCl. The aqueous layer was extracted with CH₂Cl₂ and the combined organic layers were washed with brine and dried over anhydrous Na₂SO₄. Solvent was removed in vacuo and the resulting residue was purified by flash chromatography on silica gel (Hexane/EA=3:1) afforded desired compound 0.25 g, 44% yield.

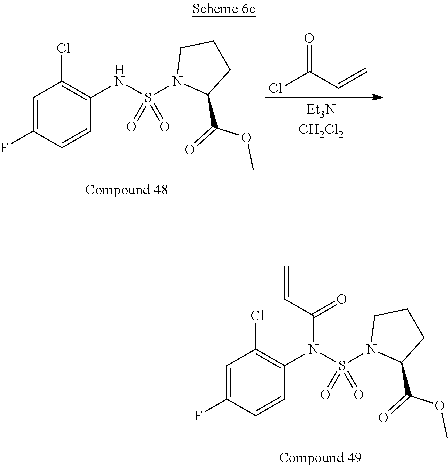

Compound 49 was produced by following Scheme 6c using Compound 48 as the starting material. Yield of Compound 49 was 13%. Colorless liquid.

¹H-NMR (300 MHz, CDCl₃): δ 7.39 (ddd, 1H), 7.29-7.25 (m, 1H), 7.13-7.06 (m, 1H), 6.49 (ddd, 1H), 5.74 (dt, 2H), 4.97 (dd, 1H), 3.80-3.51 (m, 5H), 2.38-2.27 (m, 1H), 2.16-1.99 (m, 3H). ESI-MS m/z calcd for C₁₅H₁₆ClFN₂O₅S 390.05, found 391.1 [M+H]⁺.

### 1.6.2 Synthesis of Compound 50

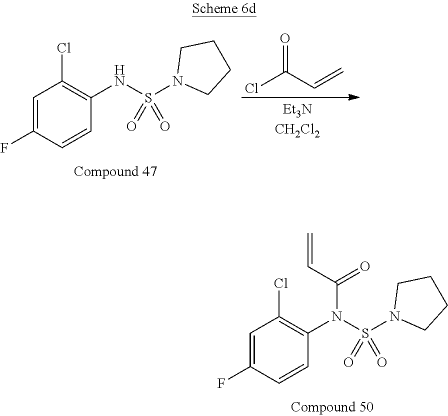

Compound 50 was produced by following Scheme 6c using Compound 47 as the starting material. Yield of Compound 50 was 21%. White solid.

¹H-NMR (400 MHz, CDCl₃): δ 7.43 (dd, 1H), 7.29-7.26 (m, 1H), 7.12 (td, 1H), 6.50 (d, 1H), 5.82 (dd, 1H), 5.68 (d, 1H), 3.72-3.59 (m, 4H), 2.02-1.95 (m, 4H). ESI-MS m/z calcd for C₁₃H₁₄ClFN₂O₃S 332.04, found 333.1 [M+H]⁺.

### 1.6.3 Synthesis of Compound 52

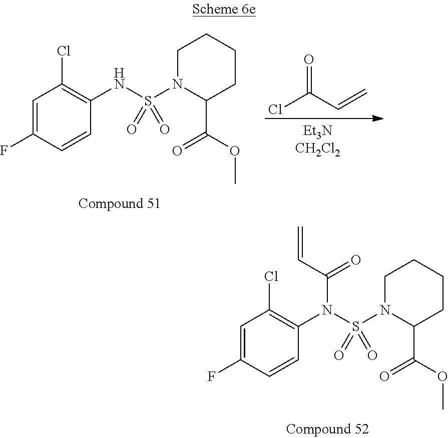

Compound 52 was produced by following Scheme 6c using compound 51 as the starting material. Yield of Compound 52 was 47%. Colorless liquid.

¹H-NMR (400 MHz, CDCl₃): δ 7.43 (ddd, 1H), 7.30-7.28 (m, 1H), 7.12-7.07 (m, 1H), 6.48 (td, 1H), 5.80-5.73 (m, 1H), 5.71-5.66 (m, 1H), 5.00 (dd, 1H), 3.90 (dd, 1H), 3.77 (s, 3H), 3.53-3.43 (m, 1H), 2.23 (t, 1H), 1.95-1.92 (m, 1H), 1.84-1.66 (m, 2H), 1.59-1.52 (m, 1H), 1.35-1.26 (m, 1H). ESI-MS m/z calcd for C₁₆H₁₈ClFN₂O₅S 404.06, found 405.1 [M+H]⁺.

### 1.7 Synthesis of Compound 57

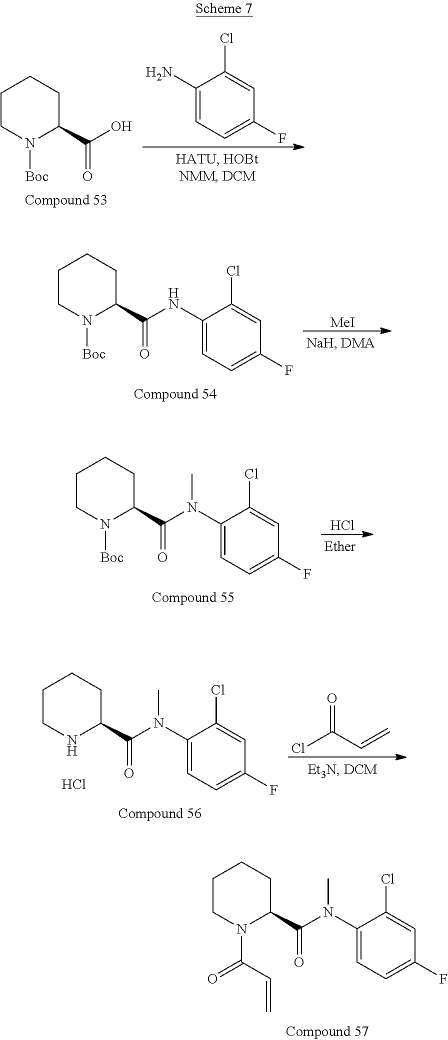

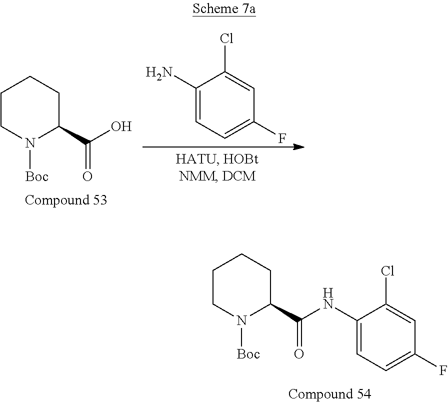 [SMILES: CC(C)(C)OC(=O)N1CCCC[C@H]1C(=O)O ; Nc1ccc(F)cc1Cl ; CC(C)(C)OC(=O)N1CCCC[C@H]1C(=O)Nc1ccc(F)cc1Cl]

To a magnetically stirred solution of carboxylic acid (1.0 g) in dichloromethane (10 mL) under an atmosphere of nitrogen was added HATU (2.1 g), HOBt (743 mg) and N-methylmorpholine (NMM) (1.2 mL) at 25° C. After the mixture was stirred at 25° C. for 10 minutes, aniline (446 μl ) was added to the mixture in one potion. The reaction mixture was stirred for another 16 hours. The resulting mixture was extracted with dichloromethane (3×50 mL). The combined extracts were washed with brine, dried over anhydrous sodium sulfate, filtered, and concentrated. The residue thus obtained was purified by flash chromatography on silica gel with EA/hexane (1/8) to give Compound 54 (55 mg, 4% yield) as a solid.

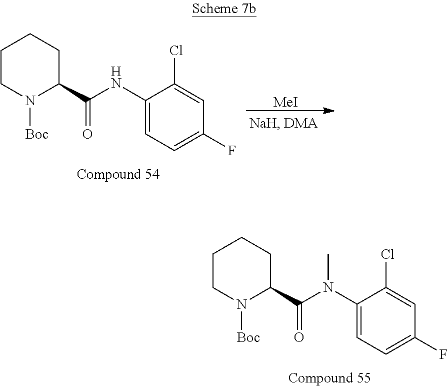 [SMILES: "MeI" ; CC(C)(C)OC(=O)N1CCCC[C@H]1C(=O)Nc1ccc(F)cc1Cl ; CN(C(=O)[C@@H]1CCCCN1C(=O)OC(C)(C)C)c1ccc(F)cc1Cl]

To a solution of mixture Compound 54 (55.0 mg, 0.15 mmol) and NaH (4.5 mg, 0.19 mmol) in DMA (2 mL) at 0° C., stirred for 10 minutes. MeI (11.5 μl, 0.19 mmol) was added and the crude was stirred for 4 hours. The crude was concentrated to remove the DMA to give the crude Compound 55. The crude Compound 55 was used in the next step without further purification.

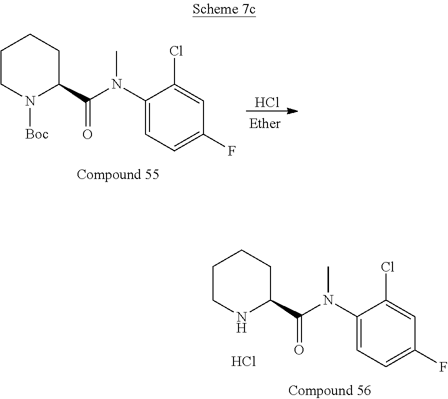 [SMILES: CN(C(=O)[C@@H]1CCCCN1C(=O)OC(C)(C)C)c1ccc(F)cc1Cl ; Cl ; CN(C(=O)[C@@H]1CCCCN1)c1ccc(F)cc1Cl ; Cl]

To a solution of mixture the crude Compound 55 and 2N HCl in ether at 25° C., stirred for 3 hours. The crude product was concentrated to remove the HCl to give the crude Compound 56. The crude Compound 56 was used in the next step without further purification.

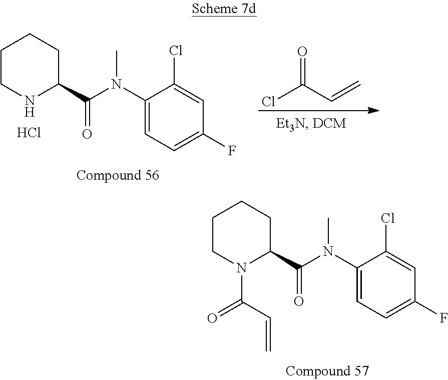

To a magnetically stirred solution of crude Compound 56 in CH₂Cl₂ (8 mL) under an atmosphere of nitrogen was added Et₃N (50.0 μl) and acyl chloride (35.0 μl ) at 25° C., and continued to stir for 1 hour. The resulting mixture was extracted with EA and NaHCO_{3(aq.)} The extracts were combined and dried over anhydrous MgSO₄, filtered, and concentrated. The residue thus obtained was purified by flash chromatography on silica gel with EA/hexane (1/8) to give Compound 57 (5 mg, 10% total yield) as a yellow oil.

¹H-NMR (400 MHz, CDCl₃): δ 7.78-7.75 (m, 1H), 7.18 (dd, 1H), 7.04-7.00 (m, 1H), 6.40 (dd, 1H), 6.23 (d, 1H), 5.67 (d, 1H), 5.05-5.03 (m, 1H), 3.90-3.74 (m, 2H), 3.17 (s, 3H), 1.82-1.26 (m, 6H). ESI-MS m/z calcd for C₁₆H₁₈ClFN₂O₂324.10, found 347.1 [M+Na]⁺.

### 1.8 Synthesis of Compound 61

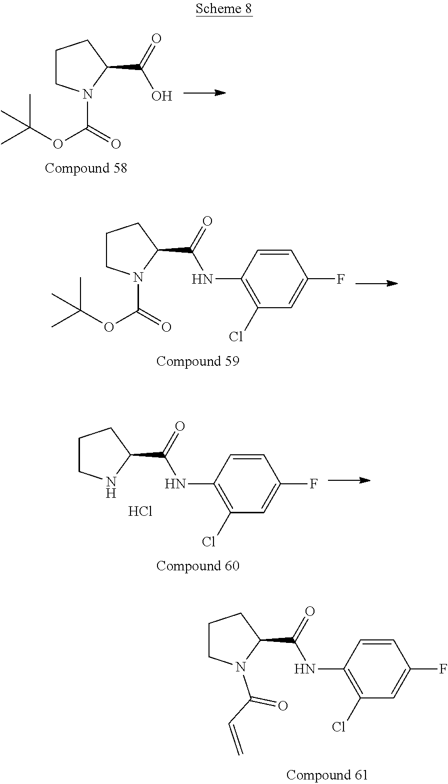 [SMILES: CC(C)(C)OC(=O)N1CCC[C@H]1C(=O)O ; CC(C)(C)OC(=O)N1CCC[C@H]1C(=O)Nc1ccc(F)cc1Cl ; O=C(Nc1ccc(F)cc1Cl)[C@@H]1CCCN1 ; Cl ; C=CC(=O)N1CCC[C@H]1C(=O)Nc1ccc(F)cc1Cl]

N-Boc-L-proline, Compound 58, (600 mg, 2.79 mmol), N-(3-Dimethylaminopropyl)-N′-ethylcarbodiimide hydrochloride (EDCI, 427 mg, 2.23 mmol), 1-Hydroxybenzotriazole hydrate (HOBt, 301 mg, 2.23 mmol) were dissolved in DCM (5.0 mL)and treated with N-methylmorpholine (NMM, 0.30 mL, 2.79 mmol),3-chloro-4-fluoroaniline (0.22 mL, 1.86 mmol) under 0° C. The reaction was reacted for 24 hours at room temperature. Then, the solution was treated with cold water, washed with brine, the organic layer dried with MgSO₄ and the solvent removed under reduced pressure. The crude product was purified by flash chromatography over silica gel with DCM/Hexane=½ to EA/Hexane=⅕, Compound 59 was obtained as a red liquid (135 mg, 21%).

Compound 59 (135 mg, 0.39 mmol) was then dissolved in MeOH (1.5 mL) and treated with 2 N HCl in ether (1.0 mL, 1.95 mmol) at room temperature. When the reaction was completed (monitored by TLC), the solvent was removed under reduced pressure to give Compound 60 (red liquid, 87 mg, 80%).

Compound 60 (87 mg, 0.31 mmol), N-(3-Dimethylaminopropyl)-N′-ethylcarbodiimide hydrochloride (EDCI, 71 mg, 0.37 mmol), 1-hydroxybenzotriazole hydrate (HOBt, 50 mg, 0.37 mmol) were dissolved in DCM (1.5 mL) and treated with N-methylmorpholine (NMM, 0.052 mL, 0.47 mmol), acrylic acid (0.032 mL, 0.47 mmol) under 0° C. The reaction was reacted for 7 hours under room temperature. Then, the solution was treated with cold water, washed with brine, the organic layer dried with MgSO₄ and the solvent removed under reduced pressure. The crude product was purified by flash chromatography over silica gel with EA/Hexane=½ to 2/1 to give title Compound 61 as a colorless liquid (10 mg, 11%).

¹H-NMR (300 MHz, CDCl₃): δ 9.55 (s, 1H), 8.22 (dd, 1H), 7.10 (dd, 1H), 7.00-6.91 (m, 1H), 6.55-6.45 (m, 1H), 6.43 (s, 1H), 5.88-5.76 (m, 1H), 4.90 (d, 1H), 3.80-3.69 (m, 1H), 3.65-3.53 (m, 1H), 2.67-2.54 (m, 1H), 2.25-2.00 (m, 2H), 1.99-1.80 (m, 1H). ESI-MS m/z calcd for C₁₄H₁₄ClFN₂O₂ 296.07, found 297.73 [M+H]⁺.

### 1.9 Synthesis of Compound 65

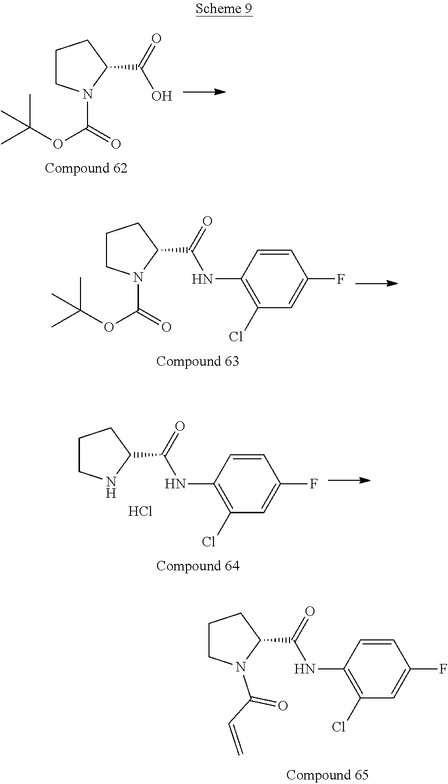

Compound 65 was produced by following Scheme 9 using compound 62 as the starting material. Yield of Compound 65 was 4%. Colorless liquid.

¹H-NMR (400 MHz, CDCl₃): δ 9.56 (s, 1H), 8.21 (dd, 1H), 7.10 (dd, 1H), 7.00-6.92 (m, 1H), 6.50 (d, 1H), 6.48 (d, 1H), 5.80 (dd, 1H), 4.80 (d, 1H), 3.77-3.70 (m, 1H), 3.64-3.53 (m, 1H), 2.64-2.55 (m, 1H), 2.20-2.02 (m, 2H), 1.93-1.80 (m, 1H). ESI-MS m/z calcd for C₁₄H₁₄ClFN₂O₂ 296.07, found 297.73 [M+H]⁺.

### 1.10 Synthesis of Compound 70

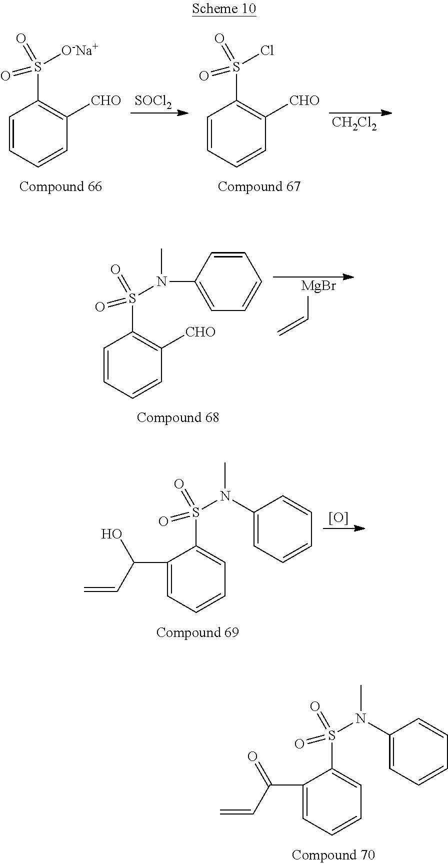

Sodium 2-formylbenzenesulfonate (Compound 66) (5.0 g, 24.04 mmol) was suspended in thionyl chloride (20 ml) and dimethylformamide (0.3 mL) was added and the mixture was heated at 100° C. for 3 minutes. The mixture was allowed to cool. The mixture was then cooled in an ice bath and water was added slowly until there was no further reaction, and with the formation of a white precipitate. The mixture was extracted with Et₂O. The combined organics were dried over MgSO₄, filtrated and evaporated under reduced pressure. And followed by column chromatography (10% ethyl acetate-hexane) afforded 2-formylbenzene-1-sulfonyl chloride (Compound 67). Yellowish liquid, yield 2.29 g, 45%.

To a solution of 2-formylbenzene-1-sulfonyl chloride (Compound 67) (2.2 g, 10.75 mmol) in dichloromethane (50 mL) and pyridine 2 mL was added N-methylaniline (1.45, 13.5 mmol). The reaction stirred at room temperature for 16 hours, after which the solvent was removed in vacuo. The resulting residue was diluted in EA 60 mL and H₂O 80 mL, the organic layers were separated, and the aqueous layer was extracted with EA, dried (sodium sulfate), filtered and concentrated, then purified by column chromatography (50% ethyl acetate-hexane) afforded 2-formyl-N-methyl-N-phenylbenzenesulfonamide (compound 68) (2.3 g, 77% yield).

A solution of 2-formyl-N-methyl-N-phenylbenzenesulfonamide (Compound 68) (200 mg, 0.73 mmol) in 5 mL of THF was added to 2 mL of 14% (H₂C═CH₂) MgBr in THF. The resulting suspension was stirred for 3.5 h and then quenched with 10 mL of saturated NH₄Cl solution. The solution was extracted with Et₂O (2×20 mL) and separated. The combined organic extracts were washed with water and saturated NaCl solution. After drying (MgSO₄), the solution was evaporated to give an orange liquid. Then purified by column chromatography (50% ethyl acetate-hexane) afforded N-[2-(1-hydroxy-allyl)-phenyl]-methanesulfonamide (Compound 69) (0.18 g, 81% yield).

A solution of 0.18 g (0.6 mmol) of N-[2-(1-Hydroxy-allyl)-phenyl]-methanesulfonamide (Compound 69) in 10 ml of dichloromethane was added dropwise to a solution of 0.33 g (0.78 mmol) of 1,1,1-triacetoxy-1,1-dihydro-1,2-benziodoxol-3(1H)-one at room temperature. After stirring at room temperature for 2 hours, the reaction mixture was added to a mixture of saturated aqueous sodium bicarbonate solution and sodium thiosulfate and extracted three times with dichloromethane. The organic phase was dried, filtered and concentrated. The compound was purified by column chromatography (30% ethyl acetate-hexane) afforded Compound 70 (0.15 g, yield 83%).

Compound 70, ¹H-NMR (400 MHz, CDCl₃): δ 7.88-7.71 (m, 2H), 7.63-7.56 (m, 1H), 7.42-7.16 (m, 6H), 6.68-6.60 (m, 1H), 6.09-6.06 (d, 1H), 5.81-5.77 (d, 1H), 3.21 (s, 3H), ESI-MS m/z calcd for C₁₆H₁₅NO₃S 301.08, found 302.1 [M+H]⁺

### Example 2 Characterization of Compounds of Example 1

### 2.1 Effects of the Compound of Example 1 on TLR4 Activity and Cell Viability

Stimulation of the Toll-like receptor 4 (TLR4) was determined by assessing activation of the transcription factor NF-κB in HEK293 cells that were engineered to express TLR4. Assessment of TLR4 stimulation was based on the use of an NE-κB-inducible secreted alkaline phosphatase (SEAP) reporter system in which the SEAP reporter was under the control of a promoter inducible by NF-κB. Thus, the degree of activation of TLR4 can be indirectly quantified spectrophotometrically by measuring the amount of the SEAP reporter that is produced.

The TLR4-expressing cells were plated in HEK-Blue Detection (Invivogen, San Diego, Calif.) medium in a 96-well plate (25,000-50,000 cells/well). The cells were stimulated with lipopolysaccharides (LPS) alone or in combination with the compound of Example 1. Cell culture without LPS and the compound of Example 1 was defined as baseline control. To test whether the compound of Example 1 could block that activation of the TLR4, the cells were treated with the designated compound (10, 20 or 100 μM) and LPS (final conc. 10 ng/ml/well). After an incubation period (16-24 hours) at 37° C. in a CO₂ incubator, the signals of individual samples in 96-well plates were detected at O.D._{650 nm} with a Molecular Devices SpectraMax i3 Imaging Cytometer. The TLR4 activity was expressed in percentage of LPS alone-stimulated cells minus baseline control.

The cell viability assay was performed by the MTT method. Cells were incubated with 45 μl of 5 mg/ml MTT for 1 hour at 37° C., and 150 μl of DMSO was added at the end of culture to dissolve the crystals. The signals of individual samples in 96-well plates were detected at O.D._{570 nm} with a Molecular Devices SpectraMax i3 Imaging Cytometer. Cell viability was expressed in percentage of viable LPS-stimulated cells. Results are summarized in Table 1.

TABLE 1
Effects of the compounds of example 1
on TLR-4 activity and cell viability

| | | |
|---|---|---|
| | TLR-4 | Cell |
| Test Con. | inhibition (%) | Viability (%) |

| Compound # | (μM) | mean | SD | mean | SD |
|---|---|---|---|---|---|
| 3 | 100 | 6.8 | 1.7 | ND | ND |
| 6 | 100 | 17.8 | 0.1 | ND | ND |
| 12 | 10 | 98.9 | 1.6 | 72 | 1.4 |
| 13 | 20 | 10.9 | 4.3 | 86.6 | 2.8 |
| 15 | 10 | 100.0 | 0 | 84.1 | 1.1 |
| 16 | 20 | 18.0 | 6.1 | 96.5 | 1.7 |
| 19 | 10 | 100.0 | 0 | 80 | 0.9 |
| 20 | 10 | 60.1 | 2.1 | 90.4 | 0.3 |
| 22 | 10 | 63.0 | 3.9 | 109.9 | 1.9 |
| 24 | 10 | 100.0 | 0 | 88.5 | 0.7 |
| 26 | 10 | 10.7 | 6.6 | 86.8 | 0.9 |
| 28 | 10 | 99.6 | 0.3 | 79.0 | 1.1 |
| 30 | 10 | 94.8 | 2.3 | 94.0 | 0.7 |
| 32 | 10 | 100.0 | 0 | 84.5 | 0.3 |
| 34 | 10 | 100.0 | 0 | 63.0 | 1.3 |
| 36 | 10 | 67.3 | 7.2 | 107.0 | 1.0 |
| 38 | 10 | 100.0 | 0 | 61.6 | 2.3 |
| 40 | 10 | 100.0 | 0 | 85.5 | 3.2 |
| 42 | 10 | 100.0 | 0 | 72.4 | 1.8 |
| 44 | 10 | 100.0 | 0 | 81.4 | 2.2 |
| 45 | 10 | 65.6 | 2.3 | 100.7 | 1.0 |
| 49 | 10 | 76.2 | 3.1 | 108.5 | 1.5 |
| 50 | 10 | 76.0 | 1.7 | 85.1 | 1.6 |
| 52 | 10 | 100.0 | 0 | 92.2 | 1.2 |
| 57 | 10 | 17.5 | 4.1 | 95.9 | 0.8 |
| 61 | 10 | 3.7 | 0.5 | 93.4 | 3.6 |
| 65 | 10 | 15.8 | 8.5 | 100.5 | 1.0 |
| 70 | 10 | 100.0 | 0.0 | 79.7 | 4.4 |

ND: Not determined.

It is evident from Table 1 that compounds 12, 15, 19, 24, 28, 30, 32, 34, 38, 40, 42, 44, 52 and 70 are strong antagonist of TLR-4, in which the TLR-4 activity was completely inhibited; Compounds 20, 22, 36, 45, 49, and 50 respectively possess moderate inhibitory activity toward TLR-4; whereas compounds 3, 6, 13, 16, 26, 57, 61 and 65 respectively have mild or negligible activity towards TLR-4. Furthermore, except for compounds 34 and 38, most compounds of Example 1 exhibited no or minor cytotoxicity to the tested cells, with cell viability over 80%.

2.2 Effect of the Compound of Example 1 on LPS Induced mTNF-α Release

To confirm whether the inhibitory effect of the selected compounds of example 2.1 (i.e., Compounds 12, 15, 19, 20, 22, 24, 28, 30, 32, 34, 36, 38, 40, 42, 44, 45, 49, 50, 52 and 70) translates to anti-inflammation, they were further tested to see if any of them may suppress mTNF-α secretion from lipopolysaccharide (LPS)-stimulated RAW264.7 monocytes.

To this purpose, RAW264.7 cells were seeded at 1.6×10⁵ cells/well into 96-well culture plates. Test compounds (i.e., Compounds 12, 15, 19, 20, 22, 24, 28, 30, 32, 34, 36, 38, 40, 42, 44, 45, 49, 50, 52 and 70, respectively at 10 μM) were added into the culture plates in triplicate and co-cultured at 37° C. in 5% CO₂ for 30 minutes. LPS (final conc. 10 ng/ml/well) was then added into the culture plates and the cells were incubated for another 4 hours. At the end of culture, the cultured media were harvested for mTNF-α quantification by ELISA with commercial assay kits (R&D Systems, Minneapolis, Minn.) following the manufacturer's protocol. Signals of the individual samples in 96-well plates were detected at O.D._{450 nm/570 nm} with a Molecular Devices SpectraMax i3 Imaging Cytometer. The mTNF-α activity was expressed in percentage of viable LPS-stimulated cells without test compound treatment.

The cell viability assay was performed by the MTT method, in which cells were incubated with 45 μl of 5 mg/ml MTT for 1 hour at 37° C., and 150 μl of DMSO was added at the end of culture to dissolve the crystals. The signals of individual samples in 96-well plates were detected at O.D._{570 nm} with a Molecular Devices SpectraMax i3 Imaging Cytometer. Cell viability was expressed in percentage of viable LPS-stimulated cells. Results are summarized in Table 2.

TABLE 2
Effects of the compounds of Example 1
on mTNF-α activity and cell viability

| | | |
|---|---|---|
| | mTNF-α | Cell |
| Test Con. | inhibition (%) | Viability (%) |

| Compound # | (μM) | mean | SD | mean | SD |
|---|---|---|---|---|---|
| 12 | 10 | 100.0 | 0 | 25.5 | 0.3 |
| 15 | 10 | 94.7 | 1.6 | 52.4 | 1.3 |
| 19 | 10 | 96.1 | 0.3 | 38.1 | 1.3 |
| 20 | 10 | 70.9 | 12.5 | 100.0 | 0.8 |
| 22 | 10 | 45.7 | 12.5 | 96.4 | 0.4 |
| 24 | 10 | 96.0 | 1.2 | 54.5 | 1.6 |
| 28 | 10 | 87.4 | 1.8 | 97.1 | 1.3 |
| 30 | 10 | 82.9 | 4.5 | 99.7 | 0.3 |
| 32 | 10 | 97.5 | 2.5 | 48.3 | 1.9 |
| 34 | 10 | 98.5 | 0.3 | 53.9 | 1.1 |
| 36 | 10 | 13.0 | 6.3 | 102.8 | 0.7 |
| 38 | 10 | 97.1 | 0.2 | 48.0 | 1.1 |
| 40 | 10 | 99.7 | 0.2 | 93.5 | 1.2 |
| 42 | 10 | 98.6 | 1.4 | 27.8 | 0.1 |
| 44 | 10 | 94.1 | 1.5 | 96.1 | 0.2 |
| 45 | 10 | 41.1 | 4.8 | 98.3 | 0.9 |
| 49 | 10 | 98.8 | 0.5 | 81.6 | 3.0 |
| 50 | 10 | 86.1 | 3.7 | 97.6 | 0.6 |
| 52 | 10 | 97.2 | 0.2 | 88.1 | 2.0 |
| 70 | 10 | 100.0 | 0.0 | 69.6 | 3.7 |

It was found that compounds 12, 15, 19, 24, 28, 30, 32, 34, 38, 40, 42, 44, 49, 50, 52, and 70 respectively exhibited strong inhibitory activity on LPS induced mTNF-α release from RAW264.7 cells, in which inhibition was about 85-100%; compound 20 showed markable activity (about 70% inhibition); both compounds 22 and 45 had moderate activity (about 40% inhibition), whereas compound 36 possessed minor inhibitory activity, with inhibition level being about 13%.

Furthermore, each of compounds 20, 22, 28, 30, 36, 40, 44, 45, 49, 50, and 52 exhibited negligible to no toxic activity toward RAW264.7 cells, whereas compounds 12, 15, 19, 24, 32, 34, 38, 42, and 70 respectively possessed markable to moderate cytotoxicity, in which cell viability was about 25-70%.

It will be apparent to those skilled in the art that various modifications and variations can be made to the disclosed embodiments. It is intended that the specification and examples be considered as exemplary only, with the true scope of the disclosure being indicated by the following claims and their equivalents.

1-17. (canceled)

18. A compound of Formula (I): 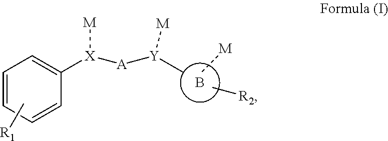
wherein: 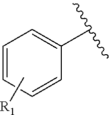
is a phenyl having at least one substituent of R₁ that is selected from the group consisting of H, alkyl, and halogen;
------ is nil or a single bond;
X and Y are independently nil, —NR′, or —CH₂—, in which R′ is H, alkyl, or a Michael acceptor;
A is —CO— or —SO₂—;
M is H, alkyl, or a Michael acceptor; 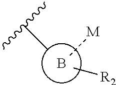
is a one or two fused ring system, carbocyclyl or heterocyclyl optionally having the Michael acceptor embedded therein or attached thereto and is optionally substituted with at least one substituent of R₂ selected from the group consisting of halogen, alkyl, haloalkoxy, —NO₂, —NRₐR_{b}, —NRₐCOR_{b}, —NRₐCOOR_{b}, —NRₐSO₂R_{b}, —NRₐSO₂NRₐR_{b}, —ORₐ, —CORₐ, —COORₐ, —SO₂Rₐ, and —SO₂NRₐR_{b};
Rₐ and R_{b} are independently H, C₁₋₂₀ alkyl, or aryl;
each alkyl and aryl is optionally substituted with at least one substituent selected from the group consisting of halogen, hydroxy, alkoxy, and phenyl; and
in cases where Y is nil, then B is not 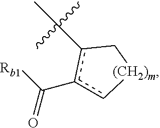
where [symbol] is a single or double bond, m is an integral between 1 to 4, and R_{b1} is H, alkyl, or alkoxy;
or a pharmaceutically acceptable salt, solvate, hydrate, co-crystal, enantiomer, or diastereoisomer thereof.

19. The compound of claim 18, wherein the Michael acceptor has the structure of 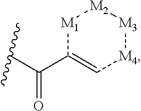
where ------ is nil or the single bond, and M₁, M₂, M₃, and M₄ are independently nil, hydrogen, or methylene.

20. The compound of claim 18, wherein the carbocyclyl is a cycloalkyl, phenyl, or aryl.

21. The compound of claim 18, wherein the heterocyclyl is a 5- or 6-membered monocyclic ring selected from the group consisting of furanyl, piperidinyl, piperazinyl, isoxazolyl, oxazolidine-2-one, and pyrrolidinyl.

22. The compound of claim 18, wherein the heterocyclyl is a bi-cyclic ring of tetrahydroquinolinyl or quinoline.

23. The compound of claim 18, wherein the compound of Formula (I) has the structure of Formula (II), 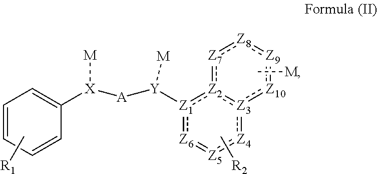
wherein: 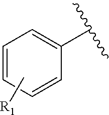
is a phenyl having at least one substituent of R₁ that is selected from the group consisting of H, alkyl, and halogen;
------ is nil or a single bond;
X and Y are independently nil, —NR′, or —CH₂—, in which R′ is H, alkyl, or M;
M is H, alkyl, or 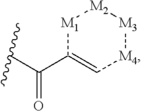
where ------ is nil or the single bond, and M₁, M₂, M₃, and M₄ are independently nil, hydrogen, or methylene;
A is —CO— or —SO₂—;
Z₁ to Z₁₀ are independently nil, C, N, or O, and are taken together to form a carbocyclyl or heterocyclyl optionally having one substituent of M as defined above and at least one substituent of R₂ that is selected from the group consisting of halogen, alkyl, haloalkoxy, —NO₂, —NRₐR_{b}, —NRₐCOR_{b}, —NRₐCOOR_{b}, —NRₐSO₂R_{b}, —NRₐSO₂NRₐR_{b}, —ORₐ, —CORₐ, —COORₐ, —SO₂Rₐ, and —SO₂NRₐR_{b}, where [symbol] is a single or double bond, and Rₐ, and R_{b} are independently H, C₁₋₂₀ alkyl or aryl, in which each alkyl and aryl is optionally substituted with at least one substituent selected from the group consisting of halogen, hydroxy, alkoxy, and phenyl; and
in cases where Y is nil, then the carbocyclyl or heterocyclyl of Z₁ to Z₁₀ is not 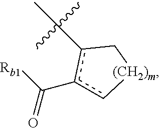
where [symbol] is the single or double bond, m is an integral between 1 to 4, and R_{b1} is H, alkyl, or alkoxy.

24. The compound of claim 23, wherein the carbocyclyl is cycloalkyl, aryl, or phenyl.

25. The compound of claim 23, wherein the heterocyclyl is a 5- or 6-membered monocyclic ring selected from the group consisting of furanyl, piperidinyl, piperazinyl, isoxazolyl, oxazolidine-2-one, and pyrrolidinyl.

26. The compound of claim 23, wherein the heterocyclyl is a bi-cyclic ring of tetrahydroquinolinyl or quinoline.

27. The compound of claim 18, wherein the compound of Formula (I) has the structure of Formula (III), 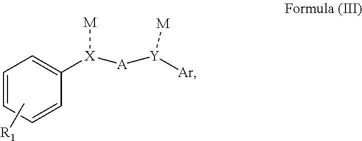
wherein: 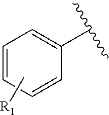
is a phenyl having at least one substituent of R₁ that is selected from the group consisting of H, alkyl, and halogen;
------ is nil or a single bond;
X and Y are independently nil, nitrogen, or carbon;
A is —CO or —SO₂—;
M is H, alkyl, or 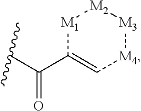
in which ------ is nil or the single bond, and M₁, M₂, M₃, and M₄ are independently nil, hydrogen, or methylene;
Ar is phenyl or heteroaryl optionally substituted with at least one substituent selected from the group consisting of H, halogen, amine, nitro, alkyl, alkenyl, alkoxy, —COOR″, —SO₂R″, or —NHCOR″, where R″ is C₁₋₄ lower alkyl or alkenyl; and
each alkyl and alkenyl is optionally substituted with at least one substituent selected from the group consisting of halogen, amine, nitro, or hydroxy.

28. The compound of claim 27, wherein the heteroaryl is tetrahydroquinolinyl or quinoline.

29. The compound of claim 18, wherein the compound is selected from the compounds delineated in Table A:

| Compound | |
|---|---|
| Number | Structure |
| 3 | 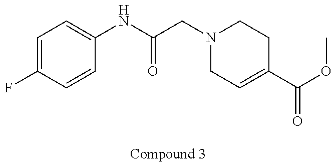 [SMILES: COC(=O)C1=CCN(CC(=O)Nc2ccc(F)cc2)CC1] |
| 6 | 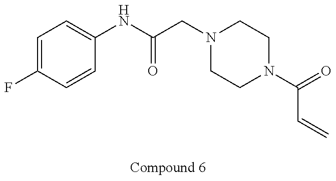 [SMILES: C=CC(=O)N1CCN(CC(=O)Nc2ccc(F)cc2)CC1] |
| 12 | 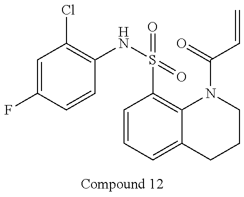 [SMILES: C=CC(=O)N1CCCc2cccc(S(=O)(=O)Nc3ccc(F)cc3Cl)c21] |
| 13 | 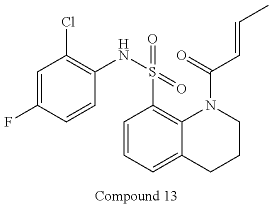 [SMILES: C/C=C/C(=O)N1CCCc2cccc(S(=O)(=O)Nc3ccc(F)cc3Cl)c21] |
| 15 | 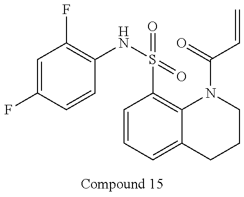 [SMILES: C=CC(=O)N1CCCc2cccc(S(=O)(=O)Nc3ccc(F)cc3F)c21] |
| 16 | 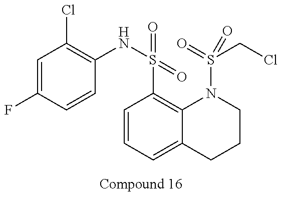 [SMILES: O=S(=O)(Nc1ccc(F)cc1Cl)c1cccc2c1N(S(=O)(=O)CCl)CCC2] |
| 19 | 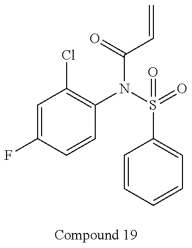 [SMILES: C=CC(=O)N(c1ccc(F)cc1Cl)S(=O)(=O)c1ccccc1] |
| 20 | 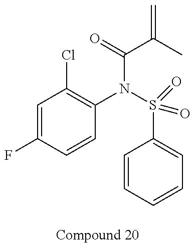 [SMILES: C=C(C)C(=O)N(c1ccc(F)cc1Cl)S(=O)(=O)c1ccccc1] |
| 22 | 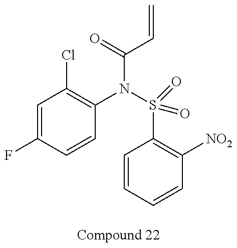 [SMILES: C=CC(=O)N(c1ccc(F)cc1Cl)S(=O)(=O)c1ccccc1[N+](=O)[O-]] |
| 24 | 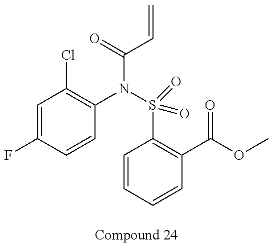 [SMILES: C=CC(=O)N(c1ccc(F)cc1Cl)S(=O)(=O)c1ccccc1C(=O)OC] |
| 26 | 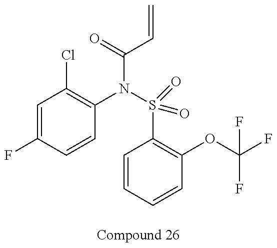 [SMILES: C=CC(=O)N(c1ccc(F)cc1Cl)S(=O)(=O)c1ccccc1OC(F)(F)F] |
| 28 | 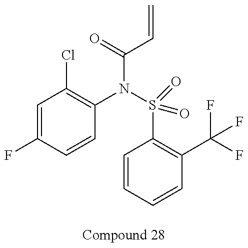 [SMILES: C=CC(=O)N(c1ccc(F)cc1Cl)S(=O)(=O)c1ccccc1C(F)(F)F] |
| 30 | 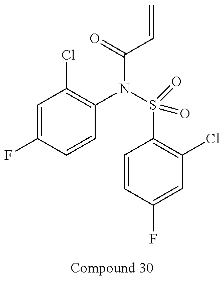 [SMILES: C=CC(=O)N(c1ccc(F)cc1Cl)S(=O)(=O)c1ccc(F)cc1Cl] |
| 32 | 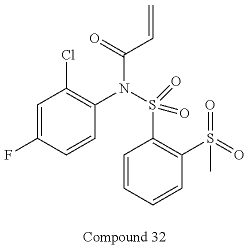 [SMILES: C=CC(=O)N(c1ccc(F)cc1Cl)S(=O)(=O)c1ccccc1S(C)(=O)=O] |
| 34 | 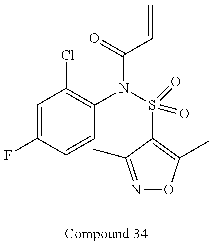 [SMILES: C=CC(=O)N(c1ccc(F)cc1Cl)S(=O)(=O)c1c(C)noc1C] |
| 36 | 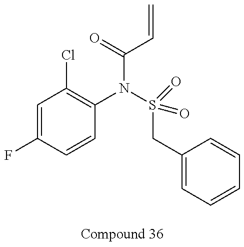 [SMILES: C=CC(=O)N(c1ccc(F)cc1Cl)S(=O)(=O)Cc1ccccc1] |
| 38 | 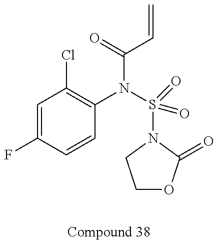 [SMILES: C=CC(=O)N(c1ccc(F)cc1Cl)S(=O)(=O)N1CCOC1=O] |
| 40 | 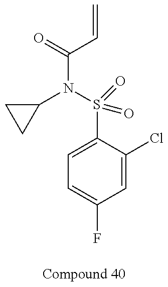 [SMILES: C=CC(=O)N(C1CC1)S(=O)(=O)c1ccc(F)cc1Cl] |
| 42 | 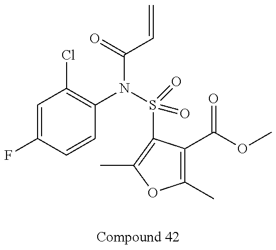 [SMILES: C=CC(=O)N(c1ccc(F)cc1Cl)S(=O)(=O)c1c(C)oc(C)c1C(=O)OC] |
| 44 | 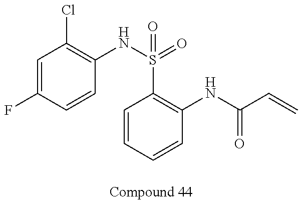 [SMILES: C=CC(=O)Nc1ccccc1S(=O)(=O)Nc1ccc(F)cc1Cl] |
| 45 | 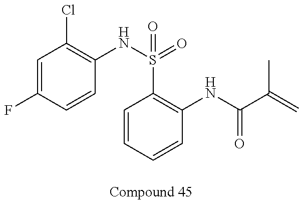 [SMILES: C=C(C)C(=O)Nc1ccccc1S(=O)(=O)Nc1ccc(F)cc1Cl] |
| 49 | 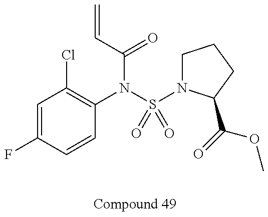 [SMILES: C=CC(=O)N(c1ccc(F)cc1Cl)S(=O)(=O)N1CCC[C@H]1C(=O)OC] |
| 50 | 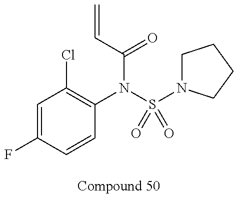 [SMILES: C=CC(=O)N(c1ccc(F)cc1Cl)S(=O)(=O)N1CCCC1] |
| 52 | 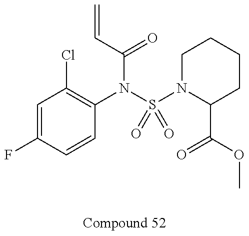 [SMILES: C=CC(=O)N(c1ccc(F)cc1Cl)S(=O)(=O)N1CCCCC1C(=O)OC] |
| 57 | 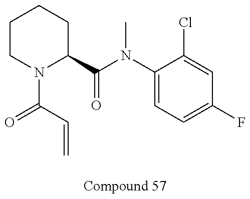 [SMILES: C=CC(=O)N1CCCC[C@H]1C(=O)N(C)c1ccc(F)cc1Cl] |
| 61 | 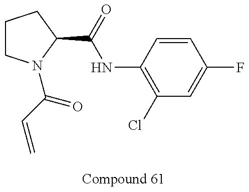 [SMILES: C=CC(=O)N1CCC[C@H]1C(=O)Nc1ccc(F)cc1Cl] |
| 65 | 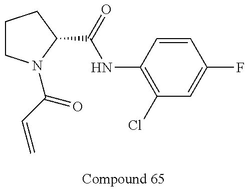 [SMILES: C=CC(=O)N1CCC[C@@H]1C(=O)Nc1ccc(F)cc1Cl] |
| 70 | 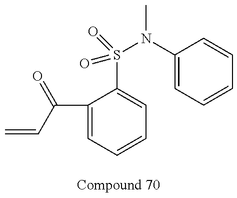 [SMILES: C=CC(=O)c1ccccc1S(=O)(=O)N(C)c1ccccc1] |

30. A pharmaceutical composition comprising an effective amount of the compound of claim 18 and a pharmaceutically acceptable carrier.

31. A method for the prophylaxis or treatment of a subject having or suspected of having a disease and/or disorder mediated by toll-like receptor 4 (TLR-4) comprising administering to the subject the pharmaceutical composition of claim 30.

32. The method of claim 31, wherein the disease and/or disorder mediated by TLR-4 is an autoimmune disease, or an inflammatory disease.

33. The method of claim 32, wherein the autoimmune disease is selected from the group consisting of multiple sclerosis, psoriasis, systemic lupus erythematosus (SLE), Type I diabetes mellitus, and Wegener's granulomatosis.

34. The method of claim 32, wherein the inflammatory disease is selected from the group consisting of asthma, allergic rhinitis, acute and chronic liver diseases, atherosclerosis, cancer, Crohn's disease, hypersensitivity lung disease, irritable bowel syndrome (IBS), inflammatory dermatoses, Sjogren's syndrome, Systemic inflammatory response syndrome (SIRS), and ulcerative colitis.
